(19) United States
(12) Patent Application Publication
(10) Pub. No.: US 2008/0171047 A1
(43) Pub. Date: Jul. 17, 2008

(54) Methods of Treating Graft Versus Host Disease

(76) Inventors: Gregg Allen Hadley, New Albany, OH (US); Riham EL-Asady, Cairo (EG)

Correspondence Address: SUGHRUE MION, PLLC, 2100 PENNSYLVANIA AVENUE, N.W., SUITE 800, WASHINGTON, DC, 20037, US

(21) Appl. No.: 11/913,409

(22) PCT Filed: Apr. 24, 2006
(86) PCT No.: PCT/US2006/015456

§ 371 Date: Mar. 28, 2008

Related U.S. Application Data
(60) Provisional application No. 60/674,209, filed on Apr. 22, 2005.

(51) Int. Cl. A61K 39/395; G01N 33/567; A61P 37/06

(52) U.S. Cl. 424/139.1; 435/7.21

(57) ABSTRACT

The present invention relates to methods for treating or preventing graft versus host disease (GVHD) in a host subject, with the methods comprising treating either the graft or the host subject or both to reduce the interaction of graft-origin, CD103-expressing cells with cells in the host. The present invention also relates to methods of screening a graft to determine the likelihood of the graft to generate GVHD.

### STATEMENT REGARDING FEDERALLY SPONSORED RESEARCH OR DEVELOPMENT

Part of the work performed during development of this invention utilized U.S. Government funds through NIH Grant No. AI36532. The U.S. Government has certain rights in this invention.

### BACKGROUND OF THE INVENTION

1. Field of the Invention

The present invention relates to methods for treating or preventing graft versus host disease (GVHD) in a host subject, with the methods comprising treating either the graft or the host subject or both to reduce the interaction of graft-origin, CD103-expressing cells with cells in the host. The present invention also relates to methods of screening a graft to determine the likelihood of the graft to generate GVHD.

2. Background of the Invention

Graft-versus-host disease (GVHD) remains the primary complication of clinical bone marrow transplantation (BMT) and a major impediment to widespread application of this important therapeutic modality. The hallmark of GVHD is infiltration of donor T lymphocytes into host epithelial compartments of the skin, intestine, and biliary tract. GVHD occurs when mature T cells, contained in the bone marrow of the graft, are transplanted into immuno-suppressed hosts. After transplantation, host antigen presenting cells (APCs) activate T cells of the graft (donor T cells) by presenting host histocompatibility antigens to the graft T-cells. Donor-derived APCs may also activate donor T cells by cross-presenting host alloantigens. The newly generated host-specific T effector (hsTeff) populations then migrate to peripheral host organs and effect target organ damage.

Intestinal injury is one of the earliest and most common features of GVHD. T cell infiltration into the intestinal epithelium not only is requisite for intestinal pathology during GVHD, but also profoundly impacts the severity and overall mortality of GVHD (10). Several lines of evidence point to a key role for host-specific CD8+ T effector populations (hsCD8eff) in the development and progression of GVHD. In experimental GVHD models, hsDB8eff cells are associated with the most severe forms of intestinal pathology and predominate at the site of intestinal injury. Selective depletion of CD8+ T cells reduces the incidence of GVHD following BMT. Depletion of CD4+ cells, on the other hand, has no effect on the incidence of GVHD.

Attention to deducing the incidence of GVHD previously focused on recognition of tissue-specific major histocompatibility complex I (MHC I)/peptide complexes (See Steinmuller, D., Immunology Today 5:234-240 (1984)). However, recent studies indicate that the gut tropism of hsTeff populations is determined, in large part, by the pattern of adhesion molecules expressed on the cell surface of the intestinal epithelium.

The CD103/β7 (herein referred to as CD103) integrin, which can be expressed on T cells, has been shown to play a critical role in targeting epithelial allografts for destruction by CD8 effectors populations (Feng, Y., D., et al., J Exp Med 196:877-886 (2002), incorporated by reference). To date, however, it has not been shown that CD103 plays a significant role in directing hsTeff-induced GVHD.

Graft-derived T cells are, however, still necessary to treat tumors and other infections in immunosuppressed patients. Thus, ablating all graft derived T cells is not considered an option for treating GVHD. Thus what is needed in the art are methods of treating or preventing T cell-mediated GVHD while still retaining the benefits and protection that graft-derived T cells confer upon the host. Such methods may exploit the fact that GVHD may be, at least in part, mediated by tissue specific expression of cell adhesion molecules, rather than MHC/peptide complexes.

### SUMMARY OF THE INVENTION

The present invention relates to methods of treating or preventing GVHD in a host subject comprising treating the graft to reduce the activity of CD103 in cells originating from the graft.

The present invention also relates to methods of treating or preventing GVHD in a host comprising treating the host to reduce the ability of host cells to interact with CD103-expressing cells originating in the graft.

The present invention also relates to methods of screening a graft to determine the likelihood that the graft will generate graft versus host disease (GVHD) after transplantation comprising determining the levels of CD103 in the graft prior to transplantation. The determined levels of CD103 in the graft are then compared to normal levels of CD103 in similar tissue to determine any differences between the graft levels of CD103 and normal levels of CD103. A difference in between the levels of CD103 may indicate the likelihood that the graft will generate GVHD.

### BRIEF DESCRIPTION OF THE DRAWINGS

FIG. 1 depicts flow cytometry data identifying cell populations using cell surface markers CD8 and CD44 in various organs in a mouse model of GVHD. Data shown on the left are dot plots of 1B2 vs. CD8 expression in lymphocyte populations. Data on the right are histograms of CD44 expression in gated 1B2+CD8+ lymphocytes. Lymphocytes were isolated from the various host compartments at 7-days post bone marrow transplant.

FIG. 2 depicts flow cytometry data showing expression of CD103 in hsCD8eff cells in the host gut in a mouse model of GVHD. Data shown are histograms of CD103 expression by gated host-specific CD8⁺ T cells (IB2⁺CD8⁺) lymphocytes. Isotype control staining is represented by shaded histograms. Numbers in top panel show the mean percentage (+SE) of intestinal CD8⁺ T cells that expressed CD103.

FIG. 3 depicts flow cytometry data showing that the expression of CD103 in hsCD8eff cells is dependent upon transforming growth factor beta (TGF-β) in a mouse model of GVHD. (A) Data shown are histograms of CD44 (left) or CD103 (right) expression by gated host-specific CD8⁺ T cells (1B2⁺CD8⁺) cells. (B) Data shown are dot plot (left) and histograms (right). The dot plot shows Thy1.1 and 1B2 expression by gated CD8⁺ lymphocytes. The histograms show CD103 expression by gated host-specific CD8⁺ T cells (1B2⁺CD8⁺) cells of either a wild type 2C (Thy1.1⁺1B2⁺ cells, upper panel) or 2C-DNR (Thy1.1⁻1B2⁺ cells, lower panel) origin. Isotype control staining is indicated by shaded histograms.

FIG. 4 depicts flow cytometry data identifying characteristics of both wild-type hsCD8eff cells and CD103 knockout hsCD8eff cells that infiltrate the gut in a mouse model of GVHD. Data shown are histograms showing expression of CD11a (A), CD62L (B), TNF-α (C), IFN-γ (D), and Annexin-V (E) by wild type (left) or CD103^{-/-} (right) host-specific CD8 cells (gated Thy1.1⁺1B2⁺ or Thy1.1⁻1B2⁺ cells, respectively) isolated from the intestinal epithelium at day 6. The shaded histograms represent isotype control staining. (F) Data shown are CSFE (carboxyfluorescein diacetate succinimidyl ester) fluorescence by wild type (left) or CD103^{-/-} (right) host-specific CD8 cells (gated Thy1.1⁺1B2⁺ or Thy1.1⁻1B2⁺ cells, respectively) isolated from the intestinal epithelium. Filled histograms show CSFE fluorescence at day 0.

FIG. 5 depicts flow cytometry data showing that expression of CD103 is correlated with retention of hsCD8eff cells in the gut in a mouse model of GVHD. (A) Data shown are dot plots of Thy1.1 vs. 1B2 expression by gated CD8⁺ lymphocytes isolated from the intestinal epithelium (upper) or spleen (lower) at the indicated time points. (B) Data shown are dot plots of Thy1.1 vs. 1B2 expression by gated CD8⁺ lymphocytes isolated from the indicated organs at day 28 post-BMT. Numbers in quadrants denote the corresponding percentages among gated CD8⁺ T cells.

FIG. 6 depicts a survival curve showing the survival rate of host mice receiving a bone marrow graft from wild-type mice and from CD103 knockout mice. The curve shows the survival of recipients of B6 bone marrow cells alone (n 4, dashed line), or in combination with CD8⁺ T cells from wild type (MST=8.2±0.8 days, n=11, dotted line) or CD103^{-/-} (MST>48.6±6.5 days, n=10, solid line) donors (P<0.001).

FIG. 7 depicts micrographs of intestinal (A) and liver (B) sections in host mice receiving a bone marrow graft from wild-type mice and from CD103 knockout mice. (A) Representative H&E sections of intestinal specimens at day 14 post-BMT from recipients of wild type (left panel) or CD103−/− (right panel) CD8⁺ T cells, at low (20×, upper panel) and high (40×, lower panel) power magnification. Arrows denote apoptotic bodies. (B) Representative H&E sections of liver specimens at day 14 post-BMT in recipients of wild type (left panel) or CD103^{-/-} (right panel) CD8⁺ T cells (20×).

FIG. 8 depicts flow cytometry data showing expression of CD103 in polyclonal CD8+ cells in the host gut in a mouse model of GVHD. Data shown are dot plots of CD103 vs. CD8 expression by gated donor (H-2K^{b+}) lymphocytes infiltrating the host intestinal epithelium.

FIG. 9 depicts a survival curve showing the survival rate of host mice receiving CD103 deficient cells. CD103-deficient cells attenuate GVHD that is mediated by CD8+ T cells primed to donor MHC I alloantigens.

FIG. 10 depicts a tumor metastasis incidence curve showing the incidence of detectable metastases in mice receiving CD8+ cells. The curve shows a percentage of mice without detectable metastases. In mice receiving CD103 deficient cells and wild-type CD8+ cells, no metastases were detectable at 10 weeks, post transplantation.

FIG. 11 depicts flow cytometry data showing depletion of CD8+ T cells after treatment of mice with anti-CD103 immunotoxin. Data shown are density plots of CD8 expression vs. CD103 expression in gated lymphocytes.

### DETAILED DESCRIPTION OF THE INVENTION

The present invention relates to methods of treating or preventing graft versus host disease (GVHD) in a host subject receiving a graft comprising reducing the activity of CD103 in cells originating from the graft. As used herein, the term “host subject” is intended to mean an animal that is receiving or has received a graft in the form of a BMT. In one embodiment, the subject is a mammal. In a more specific embodiment, the host subject is a non-human or human primate.

The graft material of the present invention may be an isograft in relation to the host, an allograft in relation to the host or even a xenograft in relation to the host. The term isograft is used as it is in art to mean a graft taken from an organism that is genetically identical to the host subject, such as an identical twin or cloned organism. The term allograft is used as it is in the art to mean a graft taken from an organism that is a member of the same species as the host subject, for example a human graft to a non-genetically identical human. The term xenograft is used as it is in the art to mean a graft from an organism that is not a member of the same species as the host subject, for example a porcine graft to a human host.

GVHD is associated with bone marrow transplant (BMT) procedures and generally occurs in an acute and chronic form. Acute GVHD will be observed within about the first 100 days post BMT, whereas chronic GVHD occurs after this initial 100 days. In addition to chronology, different clinical symptoms are also manifest in acute GVHD versus chronic GVHD. Acute GVHD is generally characterized by damage to host liver, skin, mucosa and intestinal epithelium in the host subject, although some forms of idiopathic pneumonia have also been reported. Chronic GVHD is, on the other hand, associated with damage to connective tissue as well as the organs and tissues damaged during acute GVHD in the host subject. In general, the methods of the present invention relate to therapies for either addressing GVHD that is already present in a host subject or preventing GVHD from arising in a host subject. In one embodiment, the present invention relates to methods of treating or preventing acute GVHD. In one specific embodiment, the methods relate to treating acute GVHD where the GVHD is damaging host intestinal epithelium. In other specific embodiments, the methods relate to treating acute GVHD where the GVHD is damaging at least one tissue selected from the group consisting of the host liver, the host skin, the host lung and the host mucosa. Of course, the methods may be used to treat acute GVHD where the GVHD is damaging more than tissue.

In another embodiment, the methods of the present invention relate to methods of treating or preventing chronic GVHD. In one specific embodiment, the methods relate to treating chronic GVHD where the GVHD is damaging host intestinal epithelium. In other specific embodiments, the methods relate to treating chronic GVHD where the GVHD is damaging at least one tissue selected from the group consisting of the host liver, the host skin, the host lung, the host connective tissue and the host mucosa. Of course, the methods may be used to treat acute GVHD where the GVHD is damaging more than tissue.

The term “therapy” is used to indicate a procedure or medicinal regimen designed to ameliorate one or more causes, symptoms, or untoward effects of GVHD in a host subject. The therapy can, but need not, cure the subject, i.e., remove the cause(s), or remove entirely the symptom(s) and/or untoward effect(s) of GVHD in the host subject.

“Prevention” on the other hand is intended to mean a procedure or therapy that is administered or performed prior to the onset of any symptom or diagnosis of GVHD. Thus, therapeutics may be administered in advance of any visible or detectable symptom or indication of GVHD. The prophylactic administration of the substance serves to attenuate subsequently arising symptoms or prevent symptoms from arising altogether in a host subject that may or may not be at risk.

In one embodiment of the present invention, the graft is treated, rather than the host subject. Thus, the phrase “treating the graft” is intended to mean administering a composition or performing a procedure to the graft material, where the treatment is not intended to directly affect the host organism. Of course, successful treatment of the graft will indirectly affect the host organism in that the severity of GVHD may be reduced, or even removed entirely. The methods of the invention are not limited to the location of the graft at the time the graft is treated. Thus, in one embodiment, the graft is treated prior to removal from the donor organism. In another embodiment, the graft is treated after removal from the donor organism. In yet another embodiment, the graft is treated after removal from the donor organism, but prior to transplantation into the host subject. In still another embodiment, the graft is treated after transplantation into the host organism.

In another embodiment, the host or host tissue is treated rather than the graft. “Treating the host” includes, but is not limited to, treating the host tissue that is damaged or suspected of being susceptible to damage during GVHD, such as intestinal epithelium, liver, mucosa, the lungs, skin and connective tissues thereof. Thus, the methods of the present invention may comprise administering a therapeutic regimen to a subject suffering from GVHD or to a subject that is susceptible to GVHD.

As used herein, the term “administer” and “administering” are used to mean introducing at least one compound into a subject. When administration is for the purpose of treatment, the substance is provided at, or after the onset of, a diagnosis of GVHD. The treatment serves to attenuate any symptom, or prevent additional symptoms from arising. The route of administration of the composition includes, but is not limited to, topical, transdermal, intranasal, vaginal, rectal, oral, subcutaneous intravenous, intraarterial, intramuscular, intraosseous, intraperitoneal, epidural and intrathecal.

Furthermore, the methods of treating or preventing GVHD also relate to coadministering one or more substances in addition to compounds that reduce levels of active CD103. The term “co-administer” indicates that each of at least two compounds is administered during a time frame wherein the respective periods of biological activity or effects overlap. Thus the term includes sequential as well as coextensive administration of the compounds of the present invention. And similar to administering compounds, coadministration of more than one substance can be for therapeutic and/or prophylactic purposes. If more than one substance is coadministered, the routes of administration of the two or more substances need not be the same. In addition, the term co-administer also includes administering a therapeutic regimen to the host and separately treating the graft. The scope of the invention is not limited by the identity of the substance which may be coadministered. For example, antibodies of the present invention may be coadministered with another pharmaceutically active substance, such as but not limited to, methotrexate and cyclosporine. Additional agents that may be co-administered include but are not limited to, antibodies directed to various targets, tacrolimus, sirolimus, interferons, opioids, TNF-α (tumor necrosis factor-α) binding proteins, Mycophenolate mofetil and other inhibitors of inosine monophosphate dehydrogenase (IMPDH), glucocorticoids, azathioprine and other cytostatic agents such as, but not limited to, antimetabolites and alkylating agents, to name a few. In one embodiment, the graft or donor may be pretreated by administration of immunosuppressive drugs such as cyclosporine (alone or in combination with steroids) and methotrexate prior to transplantation. For prevention, immunosuppressive therapy typically consists of combined regimens of methotrexate (MTX), cyclosporin (CsA), tacrolimus (FK 506), and/or a corticosteriod. Intravenous gamma-globulin preparations administered prophylactically have also been shown to be beneficial for the prevention of GVHD. In addition, Pentoxyfylline, a xanthine derivative capable of down-regulating TNF-α production, may be administered with cyclosporin plus either methotrexate or methylprednisolone to further decrease incidence of GVHD. Chronic GVHD may be treated with steroids such as prednisone, ozothioprine and cyclosporine. Also, antithymocyte globulin (ATG) and/or Ursodiol may be used. Thalidomide with immunosuppressive properties has shown promising results in the treatment of chronic GVHD. Similar to thalidomide, clofazimine may also be coadministered with compounds that reduce active levels of CD103. Antibody targets for co-administered antibodies include, but are not limited to, T cell receptor (TCR) and interleukin-2 (IL-2) and IL-2 receptors. Additionally, a CD(25) monoclonal antibody or anti-CD8 monoclonal antibody may be co-administered for GVHD prophylaxis.

Certain of the methods of the present invention are intended to reduce the activity of CD103 in cell originating from the graft. CD103, formerly known as αE integrin is present on both CD4+ T cells (helper T cells) and CD8+ T cells (cytolytic T cells, or CTLs) and serves as a receptor for E-cadherin. E-cadherin is an epithelial-cell specific ligand involved in cell adhesion. As used herein, “reducing the activity of CD103” is intended to mean reducing or decreasing the expression, activation or binding of CD103 on cells normally expressing the molecule. The reduction in activity may be a qualitative or quantitative reduction, and it may or may not include a complete removal of detectable activity. “Decreasing the expression” includes methods designed to decrease the production or post-translational processing of the protein, as well as methods that cleave all or a portion of the extracellular portion of CD103 such that the molecule is not able to bind to E-cadherin with the same affinity as would be normal without therapeutic intervention. By “decreasing” or “reducing” is meant a reduction in activity as compared to the same clinical situation without therapeutic intervention. Decreasing the activity of CD103 also includes methods designed to selectively destroy cells, selectively inhibit or prevent mitosis in cells that normally express CD103 as well as methods of preventing or inhibiting the retention of cells in tissues where CD103-expressing cells are known to adhere, e.g., intestinal epithelium. In one embodiment, the present invention is directed to reducing the activity of CD103 in specific cell populations within the graft. In a particular embodiment, the methods are directed to reducing the activity of CD103 in CD8+ cells that originate from the graft. In yet another embodiment, the present invention relates to methods of reducing the ability of host cells to interact with CD103-expressing cells originating in the graft.

Methods of determining a reduction or decrease in integrin, e.g., CD103, activity are well known in the art. Examples of assessing a reduction in CD103 activity include, but are not limited to, flow cytomerty, immunoassays, protein and RNA detection methods such as Western blots and Northern blots. Other means for assessing CD103 activity include, but are not limited to, assessing biological activities of cells expressing CD103 such as chemotactic, cytotoxic, enzymatic assays. Other indirect methods for assessing activities of CD103 include, for example, methods employed by the National Institute for Biological Standards and Control (NIBSC) in the United Kingdom for the quantification of interferon, cytokine and growth-factor activity. Such methods may include assessing levels of effector molecules that are secreted from cells that normally express CD103. For example, depleting or reducing the number of CD103 cells present in the graft, which is included in the definition of “reducing CD103 activity,” may be assessed by measuring certain cytokines such as, but not limited to, interferon gamma (IFN-γ) and tumor necrosis factor alpha (TNF-α), that are secreted by select CD103+ cells. CD103+ cells may also secrete chemokines and metaoproteinases. Animal models of GVHD that are predicative of efficacy in target host organisms may also be used to assess the reduction of CD103 in response to a therapeutic regimen or procedure. Still more ways of assessing CD103 activity include ex vivo assays of patient peripheral blood lymphocyte function and/or phenotype. Such methods are disclosed in Hadley et al., Transplantation 16:1418-1425 (1999), which is incorporated by reference.

In one embodiment, the methods of the present invention relate to administering antibodies or binding agents to treat the graft to reduce the activity of CD103 in cells originating from the graft. In another embodiment, the antibodies or binding agents bind to CD103. In another embodiment, the antibodies or binding agents bind to E-cadherin.

The present invention also relates to treating the graft comprising administering antagonists to transforming growth factor beta (TGF-β). In particular, the antagonists to TGF-β include, but are not limited to, antibodies or binding agents that bind TGF-β receptors. In a more specific embodiment, the antibodies or binding agents may be directed to TGF-β receptors that are expressed on CD8+ cells. In yet another embodiment, the antibodies or binding agents bind to transforming growth factor-β (TGF-β).

As used herein, the term “antibody” is used to mean immunoglobulin molecules and functional fragments thereof, regardless of the source or method of producing the fragment. As used herein, a “functional fragment” of an immunoglobulin is a portion of the immunoglobulin molecule that specifically binds to a binding target. Thus, the term “antibody” as used herein encompasses whole antibodies, such as antibodies with isotypes that include but are not limited to IgG, IgM, IgA, IgD, IgE and IgY, and even single-chain antibodies found in some animals e.g., camels, as well as fragments that specifically bind to target. Whole antibodies thereof may be monoclonal or polyclonal, and they may be humanized or chimeric. The term “monoclonal antibody” as used herein is not limited to antibodies produced through hybridoma technology. Rather, the term “monoclonal antibody” refers to an antibody that is derived from a single clone, including any eukaryotic, prokaryotic, or phage clone, and not the method by which it is produced. The term “antibody” also encompasses functional fragments of immunoglobulins, including but not limited to Fab fragments, Fab′ fragments, F(ab′)₂ fragments and Fd fragments. “Antibody” also encompasses fragments of immunoglobulins that comprise at least a portion of a V_{L} and/or V_{H} domain, such as single chain antibodies, a single-chain Fv (scFv), disulfide-linked Fvs and the like.

The antibodies used in the present invention may be monospecific, bispecific, trispecific or of even greater multispecificity. In addition the antibodies may be monovalent, bivalent, trivalent or of even greater multivalency. Furthermore, the antibodies of the invention may be from any animal origin including, but not limited to, birds and mammals. In specific embodiments, the antibodies are human, murine, rat, sheep, rabbit, goat, guinea pig, horse, or chicken. As used herein, “human” antibodies include antibodies having the amino acid sequence of a human immunoglobulin and include antibodies isolated from human immunoglobulin libraries or from animals transgenic for one or more human immunoglobulin and that do not express endogenous immunoglobulins, as described in U.S. Pat. No. 5,939,598, which is herein incorporated by reference.

The antibodies used in the present invention may be described or specified in terms of the epitope(s) or portion(s) of a polypeptide to which they recognize or specifically bind. Or the antibodies may be described based upon their ability to bind to specific conformations of the antigen, or specific modification (e.g., cleavage or chemical, natural or otherwise, modification of sequence).

The specificity of the antibodies used in present invention may also be described or specified in terms their binding affinity towards the antigen (epitope) or of by their cross-reactivity. Specific examples of binding affinities encompassed in the present invention include but are not limited to those with a dissociation constant (Kd) less than 5×10⁻² M, 10⁻² M, 5×10⁻³ M, 10⁻³ M, 5×10⁻⁴ M, 10⁻⁴ M, 5×10⁻⁵ M, 10⁻⁵ M, 5×10⁻⁶ M, 10⁻⁶ M, 5×10⁻⁷ M, 10⁻⁷ M, 5×10⁻⁸ M, 10⁻⁸ M, 5×10⁻⁹ M, 10⁻⁹ M, 5×10⁻¹⁰ M, 10⁻¹⁰ M, 5×10⁻¹¹ M, 10⁻¹¹ M, 5×10⁻¹² M, 10⁻¹² M, 5×10⁻¹³ M, 10⁻¹³ M, 5×10⁻¹⁴ M, 10⁻¹⁴ M, 5×10⁻¹⁵ M, or 10⁻¹⁵ M.

The antibodies used in the invention also include derivatives that are modified, for example, by covalent attachment of any type of molecule to the antibody such that covalent attachment does not prevent the antibody from generating an anti-idiotypic response. Examples of modifications to antibodies include but are not limited to, glycosylation, acetylation, pegylation, phosphorylation, amidation, derivatization by known protecting/blocking groups, proteolytic cleavage, linkage to a cellular ligand or other composition, such as a signaling moiety, a label etc. In addition, the antibodies may be linked or attached to solid substrates, such as, but not limited to, beads, particles, glass surfaces, plastic surfaces, ceramic surfaces, metal surfaces. Any of numerous chemical modifications may be carried out by known techniques, including, but not limited to, specific chemical cleavage, acetylation, biotinylation, farnesylation, formylation, inhibition of glycosylation by tunicamycin and the like. Additionally, the derivative may contain one or more non-classical or synthetic amino acids.

The antibodies used in the present invention may be generated by any suitable method known in the art. Polyclonal antibodies can be produced by various procedures well known in the art. For example, the antigen or fragment thereof can be administered to various host animals including, but not limited to, rabbits, goats, chickens, mice, rats, to induce the production of sera containing polyclonal antibodies specific for the antigen. Various adjuvants may be used to increase the immunological response, depending on the host species, and include but are not limited to, Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and Corynebacterium parvum. Such adjuvants are also well known in the art.

Monoclonal antibodies can be prepared using a wide variety of techniques known in the art including the use of hybridoma, recombinant, and phage display technologies, or a combination thereof. For example, monoclonal antibodies can be produced using hybridoma techniques including those known in the art and taught, for example, in Harlow et al, Antibodies: A Laboratory Manual, (Cold Spring Harbor Laboratory Press, 2nd ed. 1988); Hammerling, et al., in: Monoclonal Antibodies and T-Cell Hybridomas 563-681 (Elsevier, N.Y., 1981) (both of which are incorporated by reference).

Methods for producing and screening for specific antibodies using hybridoma technology are routine and well known in the art such as, but not limited to, immunizing a mouse, hamster, or rat. Additionally, newer methods to produce rabbit and other mammalian monoclonal antibodies may be available to produce and screen for antibodies. In short, methods of producing and screening antibodies, and the animals used therein, should not limit the scope of the invention. Once an immune response is detected, the mouse spleen is harvested and splenocytes isolated. The splenocytes are then fused by well known techniques to any suitable myeloma cells, for example cells from cell line SP2/0 available from the ATCC. Hybridomas are selected and cloned by limited dilution. The hybridoma clones can then be assayed by methods known in the art for cells that secrete antibodies capable of binding an antigen of the present invention. Ascites fluid, which generally contains high levels of antibodies, can be generated by immunizing mice with positive hybridoma clones. In addition, antibodies can be produced using a variety of alternate methods, including but not limited to bioreactors and standard tissue culture methods, to name a few.

The antibodies used the present invention can also be generated using various phage display methods known in the art. In phage display methods, functional antibody domains are displayed on the surface of phage particles which carry the polynucleotide sequences encoding them. In a particular embodiment, such phage can be utilized to display antigen binding domains expressed from a repertoire or combinatorial antibody library. Phage expressing an antigen binding domain that binds the antigen of interest can be selected or identified with the antigen of interest, such as using a labeled antigen or antigen bound or captured to a solid surface or bead. The phage used in these methods are typically filamentous phage including, but not limited to, fd and M13 binding domains expressed from phage with Fab, Fv or disulfide stabilized Fv antibody domains recombinantly fused to either the phage gene III or gene VIII protein. Examples of phage display methods that can be used to make the antibodies of the present invention include those disclosed in Brinkman et al., J. Immunol. Methods 182:41-50 (1995); Ames et al., J. Immunol. Methods 184:177-186 (1995); Kettleborough et al., Eur. J. Immunol. 24:952-958 (1994); Persic et al., Gene 187 9-18 (1997); Burton et al., Advances in Immunology 57:191-280 (1994); PCT application No. PCT/GB91/01134; PCT publications WO 90/02809; WO 91/10737; WO 92/01047; WO 92/18619; WO 93/11236; WO 95/15982; WO 95/20401; and U.S. Pat. Nos. 5,698,426; 5,223,409; 5,403,484; 5,580,717; 5,427,908; 5,750,753; 5,821,047; 5,571,698; 5,427,908; 5,516,637; 5,780,225; 5,658,727; 5,733,743 and 5,969,108, all of which are incorporated by reference.

Antibody fragments which recognize specific epitopes may be generated by known techniques. For example, Fab and F(ab′)₂ fragments of the invention may be produced by proteolytic cleavage of immunoglobulin molecules, using enzymes such as papain (to produce Fab fragments) or pepsin (to produce F(ab′)₂ fragments). F(ab′)₂ fragments contain the variable region, the light chain constant region and the CH1 domain of the heavy chain.

Other methods, such as recombinant techniques, may be used to produce Fab, Fab′ and F(ab′)₂ fragments and are disclosed in PCT publication WO 92/22324; Mullinax et al., BioTechniques 12(6):864-869 (1992); and Sawai et al., AJRI 34:26-34 (1995); and Better et al., Science 240:1041-1043 (1988), which are herein incorporated by reference. After phage selection, for example, the antibody coding regions from the phage can be isolated and used to generate whole antibodies, including human antibodies, or any other desired antigen binding fragment, and expressed in any desired host, including mammalian cells, insect cells, plant cells, yeast, and bacteria.

Examples of techniques which can be used to produce other types of fragments, such as scFvs and include those described in U.S. Pat. Nos. 4,946,778 and 5,258,498; Huston et al., Methods in Enzymology 203:46-88 (1991); Shu et al., Proc. Nat'l Acad. Sci. (USA) 90:7995-7999 (1993); and Skerra et al, Science 240:1038-1040 (1988), all of which are incorporated by reference. For some uses, including in vivo use of antibodies in humans and in vitro detection assays, it may be preferable to use chimeric, humanized, or human antibodies. A chimeric antibody is a molecule in which different portions of the antibody are derived from different animal species, such as antibodies having a variable region derived from a murine monoclonal antibody and a human immunoglobulin constant region. Methods for producing chimeric antibodies are known in the art. See e.g., Morrison, Science 229:1202 (1985); Oi et al., BioTechniques 4:214 (1986); Gillies et al., J. Immunol. Methods 125:191-202 (1989); U.S. Pat. Nos. 5,807,715; 4,816,567; and 4,816,397, all of which are herein incorporated by reference. Humanized antibodies are antibody molecules from non-human species antibody that bind the desired antigen having one or more complementarity determining regions (CDRs) from the non-human species and framework regions from a human immunoglobulin molecule. Often, framework residues in the human framework regions will be substituted with the corresponding residue from the CDR donor antibody to alter, preferably improve, antigen binding. These framework substitutions are identified by methods well known in the art, e.g., by modeling of the interactions of the CDR and framework residues to identify framework residues important for antigen binding and sequence comparison to identify unusual framework residues at particular positions. (See U.S. Pat. No. 5,585,089; Riechmann et al., Nature 332:323 (1988), both of which are herein incorporated by reference. Antibodies can be humanized using a variety of techniques known in the art including, for example, CDR-grafting (EP 239,400; PCT publication WO 91/09967; U.S. Pat. Nos. 5,225,539; 5,530,101; and 5,585,089), veneering or resurfacing (EP 592,106; EP 519,596; Padlan, Molecular Immunology 28(4/5):489-498 (1991); Studnicka et al., Protein Engineering 7(6):805-814 (1994); Roguska. et al., Proc. Nat'l. Acad. Sci. 91:969-913 (1994)), and chain shuffling (U.S. Pat. No. 5,565,332), all of which are hereby incorporated by reference.

Completely human antibodies may be particularly desirable for therapeutic treatment or diagnosis of human patients. Human antibodies can be made by a variety of methods known in the art including phage display methods described above using antibody libraries derived from human immunoglobulin sequences. See also. U.S. Pat. Nos. 4,444,887 and 4,716,111; and PCT publications WO 98/46645, WO 98/50433, WO 98/24893, WO 98/16654, WO 96/34096, WO 96/33735, and WO 91/10741; each of which is incorporated by reference.

Human antibodies can also be produced using transgenic mice which are incapable of expressing functional endogenous immunoglobulins, but which can express human immunoglobulin genes. For example, the human heavy and light chain immunoglobulin gene complexes may be introduced randomly or by homologous recombination into mouse embryonic stem cells. Alternatively, the human variable region, constant region, and diversity region may be introduced into mouse embryonic stem cells in addition to the human heavy and light chain genes. The mouse heavy and light chain immunoglobulin genes may be rendered non-functional separately or simultaneously with the introduction of human immunoglobulin loci by homologous recombination. In particular, homozygous deletion of the JH region prevents endogenous antibody production. The modified embryonic stem cells are expanded and microinjected into blastocysts to produce chimeric mice. The chimeric mice are then bred to produce homozygous offspring which express human antibodies. The transgenic mice are immunized in the normal fashion with a selected antigen. Monoclonal antibodies directed against the antigen can be obtained from the immunized, transgenic mice using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA, IgM and IgE antibodies. For an overview of this technology for producing human antibodies, see Lonberg and Huszar, Int. Rev. Immunol. 13:65-93 (1995), which is hereby incorporated by reference. For a detailed discussion of this technology for producing human antibodies and human monoclonal antibodies and protocols for producing such antibodies, see, e.g., PCT publications WO 98/24893; WO 92/01047; WO 96/34096; WO 96/33735; European Patent No. 0 598 877; U.S. Pat. Nos. 5,413,923; 5,625,126; 5,633,425; 5,569,825; 5,661,016; 5,545,806; 5,814,318; 5,885,793; 5,916,771; and 5,939,598, which are incorporated by reference.

Still another approach for generating human antibodies utilizes a technique referred to as guided selection. In guided selection, a selected non-human monoclonal antibody, e.g., a mouse antibody, is used to guide the selection of a completely human antibody recognizing the same epitope. (Jespers et al., Biotechnology 12:899-903 (1988), herein incorporated by reference).

Various methods can be used to assess the functional activity of antibodies and fragments. For example, in one embodiment where one is assaying for the ability to bind or compete with a polypeptide for binding to, for example, CD103, various immunoassays known in the art can be used, including but not limited to, competitive and non-competitive assay systems using techniques such as radioimmunoassays, ELISA (enzyme linked immunosorbent assay), “sandwich” immunoassays, immunoradiometric assays, gel diffusion precipitation reactions, immunodiffusion assays, in situ immunoassays (using colloidal gold, enzyme or radioisotope labels, for example), western blots, precipitation reactions. agglutination assays (e.g., gel agglutination assays, hemagglutination assays), complement fixation assays, immunofluorescence assays, protein A assays, and immunoelectrophoresis assays, etc. In one embodiment, antibody binding is detected by detecting a label on the primary antibody. In another embodiment, the primary antibody is detected by detecting binding of a secondary antibody or reagent to the primary antibody. In a further embodiment, the secondary antibody is labeled. Many means are known in the art for detecting binding in an immunoassay and are within the scope of the present invention.

The antibodies of the present invention must bind an antigen comprising at least a portion of the antigen of interest. The terms “antigen” and “biomarker” are also used interchangeably, as they relate the antibodies and the methods of use described herein. Thus the term “antigen” does not necessarily mean that the biomarkers of the present invention elicit an immune response. In one specific embodiment, the antigen is a polypeptide of comprising an amino acid sequence at least 80%, 85%, 90%, 95%, 97%, 98%, 99% or 100% identical to the amino acid sequence of SEQ ID NO:1, below.

```
(SEQ ID NO:1)
      1 mwlfhtllci aslallaafn vdvarpwltp kggapfvlss llhqdpstnq twllvtsprt
    61 krtpgplhrc slvqdeilch pvehvpipkg rhrgvtvvrs hhgvliciqv lvrrphslss
  121 eltgtcsllg pdlrpqaqan ffdlenlldp darvdtgdcy snkegggedd vntarqrral
  181 ekeeeedkee eedeeeeeag teiaiildgs gsidppdfqr akdfisnmmr nfyekcfecn
  241 falvqyggvi qtefdlrdsq dvmaslarvq nitqvgsvtk tasamqhvld siftsshgsr
  301 rkaskvmvvl tdggifedpl nlttvinspk mqgverfaig vgeefksart arelnliasd
  361 pdethafkvt nymaldglls klryniisme gtvgdalhyq laqigfsaqi lderqvllga
  421 vgafdwsgga llydtrsrrg rflnqtaaaa adaeaaqysy lgyavavlhk tcslsyvaga
  481 pqykhhgavf elqkegreas flpvlegeqm gsyfgselcp vdidmdgstd fllvaapfyh
  541 vhgeegrvyv yrlseqdgsf slarilsghp gftnarfgfa maamgdlsqd kltdvaigap
  601 legfgaddga sfgsvyiyng hwdglsasps qrirastvap glqyfgmsma ggfdisgdgl
  661 aditvgtlgq avvfrsrpvv rlkvsmaftp salpigfngv vnvrlcfeis svttasesgl
  721 reallnftld vdvgkqrrrl qcsdvrsclg clrewssgsq lcedlllmpt egelceedcf
  781 snasvkvsyq lqtpegqtdh pqpildryte pfaifqlpye kacknklfcv aelqlattvs
  841 qqelvvgltk eltlninltn sgedsymtsm alnyprnlql krmqkppspn iqcddpqpva
  901 svlimncrig hpvlkrssah vsvvwqleen afpnrtadit vtvtnsnerr slanethtlq
  961 frhgfvavls kpsimyvntg qglshhkefl fhvhgenlfg aeyqlqicvp tklrglqvaa
1021 vkkltrtqas tvctwsqera cayssvqhve ewhsvscvia sdkenvtvaa eiswdhseel
1081 lkdvtelqil geisfnksly eglnaenhrt kitvvflkde kyhslpiiik gsvggllvli
1141 vilvilfkcg ffkrkyqqln lesirkaqlk senlleeen
```

In another specific embodiment, the antigen is a polypeptide of comprising an amino acid sequence at least 80%, 85%, 90%, 95%, 97%, 98%, 99% or 100% identical to residues 2-1179 of the amino acid sequence of SEQ ID NO:1. In another specific embodiment, the antigen is a polypeptide of comprising an amino acid sequence at least 80%, 85%, 90%, 95%, 97%, 98%, 99% or 100% identical to residues 19-1179 of the amino acid sequence of SEQ ID NO:1. In another specific embodiment, the antigen is a polypeptide of comprising an amino acid sequence at least 80%, 85%, 90%, 95%, 97%, 98%, 99% or 100% identical to residues 19-176 of the amino acid sequence of SEQ ID NO:1. In another specific embodiment, the antigen is a polypeptide of comprising an amino acid sequence at least 80%, 85%, 90%, 95%, 97%, 98%, 99% or 100% identical to residues 177-1179 of the amino acid sequence of SEQ ID NO:1. In another specific embodiment, the antigen is a polypeptide of comprising an amino acid sequence at least 80%, 85%, 90%, 95%, 97%, 98%, 99% or 100% identical to residues 144-198 of the amino acid sequence of SEQ ID NO:1. In another specific embodiment, the antigen is a polypeptide of comprising an amino acid sequence at least 80%, 85%, 90%, 95%, 97%, 98%, 99% or 100% identical to residues 199-390 of the amino acid sequence of SEQ ID NO:1. In another specific embodiment, the antigen is a polypeptide of comprising an amino acid sequence at least 80%, 85%, 90%, 95%, 97%, 98%, 99% or 100% identical to residues 201-378 of the amino acid sequence of SEQ ID NO:1. In another specific embodiment, the antigen is a polypeptide of comprising an amino acid sequence at least 80%, 85%, 90%, 95%, 97%, 98%, 99% or 100% identical to residues 510-565 of the amino acid sequence of SEQ ID NO:1. In another specific embodiment, the antigen is a polypeptide of comprising an amino acid sequence at least 80%, 85%, 90%, 95%, 97%, 98%, 99% or 100% identical to residues 574-634 of the amino acid sequence of SEQ ID NO:1.

As used herein, “identity” as it relates to amino acid sequence or polynucleotide sequences is a measure of the identity of nucleotide sequences or amino acid sequences compared to a reference nucleotide or amino acid sequence, usually a wild-type sequence. In general, the sequences are aligned so that the highest order match is obtained. “Identity” per se has an art-recognized meaning and can be calculated using published techniques. (See, e.g., Computational Molecular Biology, Lesk, A. M., ed., Oxford University Press, New York (1988); Biocomputing: Informatics And Genome Projects, Smith, D. W., ed., Academic Press, New York (1993); Computer Analysis of Sequence Data, Part I, Griffin, A. M., and Griffin, H. G., eds., Humana Press, New Jersey (1994); von Heinje, G., Sequence Analysis In Molecular Biology, Academic Press (1987); and Sequence Analysis Primer, Gribskov, M. and Devereux, J., eds., M Stockton Press, New York (1991)). While several methods exist to measure identity between two polynucleotide or polypeptide sequences, the term “identity” is well known in the art (Carillo, H. & Lipton, D., Siam J Applied Math 48:1073 (1988)). Methods commonly employed to determine identity or similarity between two sequences include, but are not limited to, those disclosed in Guide to Huge Computers, Martin J. Bishop, ed., Academic Press, San Diego (1994) and Carillo, H. & Lipton, D., Siam J Applied Math 48:1073 (1988). Computer programs may also contain methods and algorithms that calculate identity and similarity. Examples of computer program methods to determine identity and similarity between two sequences include, but are not limited to, GCS program package (Devereux, J., et al., Nucleic Acids Research 12(i):387 (1984)), BLASTP, BLASTN, FASTA (Atschul, S. F., et al., J Molec Biol 215:403 (1990)).

A polypeptide having an amino acid sequence at least, for example, about 95% “identical” to a reference nucleotide sequence encoding a peptide of interest, for example CD103 or an antibody to CD103, is understood to mean that the amino acid sequence of the peptide is identical to the reference sequence except that the amino acid sequence may include up to about five mutations per each 100 amino acids of the reference peptide sequence encoding the anti-CD103 peptide being used as the reference sequence. In other words, to obtain a polypeptide having an amino acid sequence at least about 95% identical to a reference amino acid sequence, up to about 5% of the amino acids in the reference sequence may be deleted or substituted with another amino acid, or a number of amino acids up to about 5% of the total amino acids in the reference sequence may be inserted into the reference sequence. These mutations of the reference sequence may occur at the N- or C-terminal positions of the reference amino acid sequence or anywhere between those terminal positions, interspersed either individually among amino acids in the reference sequence or in one or more contiguous groups within the reference sequence.

The present invention also relates to isolated nucleic acids encoding the antibodies or binding agents of the present invention. As used herein, “isolated” as it relates to a polypeptide or nucleic acid molecule, is used to mean a polypeptide or nucleic acid molecule that has been removed from its native environment. For example, polypeptides that have been removed or purified from cells are considered isolated. In addition, recombinantly produced polypeptides molecules contained in recombinant host cells are considered isolated for the purposes of the present invention. Similarly, recombinant DNA molecules contained in a vector are considered isolated for the purposes of the present invention. Further examples of isolated DNA molecules include, but are not limited to, recombinant DNA molecules maintained in recombinant host cells or purified (partially or substantially) DNA molecules in solution. Isolated RNA molecules include in vivo or in vitro RNA transcripts of the DNA molecules of the present invention. Isolated nucleic acid molecules according to the present invention further include such molecules produced synthetically.

Nucleic acid molecules of the present invention may be in the form of RNA, such as but not limited to mRNA, or in the form of DNA, including, for instance, cDNA and genomic DNA obtained by cloning or produced synthetically. The DNA may be, but is not limited to, double-stranded or single-stranded. Single-stranded DNA or RNA may be the coding strand, also known as the sense strand, or it may be the non-coding strand, also referred to as the anti-sense strand.

Using the information provided herein, a nucleic acid molecule of the present invention encoding an antibody specific for CD103 biomarker may be obtained using standard cloning and screening procedures, such as those for cloning cDNAs using mRNA as starting material.

Nucleotide sequences can be determined using an automated DNA sequencer (such as the Model 373 from Applied Biosystems, Inc.). Therefore, as is known in the art for any DNA sequence determined by this automated approach, any nucleotide sequence determined herein may contain some errors. Nucleotide sequences determined by automation are typically at least about 90% identical, more typically at least about 95% to at least about 99.9% identical to the actual nucleotide sequence of the sequenced DNA molecule. The actual sequence can be more precisely determined by other approaches including manual DNA sequencing methods well known in the art. As is also known in the art, a single insertion or deletion in a determined nucleotide sequence compared to the actual sequence will cause a frame shift in translation of the nucleotide sequence such that the predicted amino acid sequence encoded by a determined nucleotide sequence will be completely different from the amino acid sequence actually encoded by the sequenced DNA molecule, beginning at the point of such an insertion or deletion.

By a polynucleotide having a nucleotide sequence at least, for example, 95% “identical” to a reference nucleotide sequence of the present invention, it is intended that the nucleotide sequence of the polynucleotide is identical to the reference sequence except that the polynucleotide sequence may include up to five point mutations per each 100 nucleotides of the reference nucleotide sequence encoding an antibody to, for example, CD103. In other words, to obtain a polynucleotide having a nucleotide sequence at least 95% identical to a reference nucleotide sequence, up to 5% of the nucleotides in the reference sequence may be deleted or substituted with another nucleotide, or a number of nucleotides up to 5% of the total nucleotides in the reference sequence may be inserted into the reference sequence. These mutations of the reference sequence may occur at the 5′ or 3′ terminal positions of the reference nucleotide sequence or anywhere between those terminal positions, interspersed either individually among nucleotides in the reference sequence or in one or more contiguous groups within the reference sequence.

As a practical matter, whether any particular nucleic acid molecule or polypeptide is at least 80%, 85%, 90%, 92%, 95%, 96%, 97%, 98% or 99% identical to a nucleotide sequence of the presence invention can be determined conventionally using known computer programs. A preferred method for determining the best overall match between a query sequence (a sequence of the present invention) and a subject sequence, also referred to as a global sequence alignment, can be determined using the FASTDB computer program based on the algorithm of Brutlag et al. (Comp. App. Biosci. 6:237-245 (1990)). In a sequence alignment the query and subject sequences are both DNA sequences. An RNA sequence can be compared by converting U's to T's. The result of said global sequence alignment is in percent identity. Parameters that may be used in a FASTDB alignment of DNA sequences to calculate percent identity include, but are not limited to: Matrix=Unitary, k-tuple=4, Mismatch Penalty=1, Joining Penalty=30, Randomization Group Length=0, Cutoff Score=1, Gap Penalty=5, Gap Size Penalty 0.05, Window Size=500 or the length of the subject nucleotide sequence, whichever is shorter.

If the subject sequence is shorter than the query sequence because of 5′ or 3′ deletions, not because of internal deletions, a manual correction can be made because the FASTDB program does not account for 5′ and 3′ truncations of the subject sequence when calculating percent identity. For subject sequences truncated at the 5′ or 3′ ends, relative to the query sequence, the percent identity is corrected by calculating the number of bases of the query sequence that are 5′ and 3′ of the subject sequence, which are not matched/aligned, as a percent of the total bases of the query sequence. Nucleotide matching/alignment is determined by results of the FASTDB sequence alignment. The alignment percentage is then subtracted from the percent identity, calculated by the above FASTDB program using the specified parameters, to arrive at a final percent identity score. This corrected score can be used for the purposes of the present invention. Only bases outside the 5′ and 3′ bases of the subject sequence, as displayed by the FASTDB alignment, which are not matched/aligned with the query sequence, are calculated for the purposes of manually adjusting the percent identity score.

For example, a 90 base subject sequence is aligned to a 100 base query sequence to determine percent identity. In this example, the deletions occur at the 5′ end of the subject sequence and therefore, the FASTDB alignment does not show a match/alignment of the first 10 bases at the 5′ end. The 10 unpaired bases represent 10% of the sequence (number of bases at the 5′ and 3′ ends not matched/total number of bases in the query sequence) so 10% is subtracted from the percent identity score calculated by the FASTDB program. If the remaining 90 bases were perfectly matched the final percent identity would be 90%. In another example, a 90 base subject sequence is compared with a 100 base query sequence. This time the deletions are internal deletions so that there are no bases on the 5′ or 3′ of the subject sequence which are not matched/aligned with the query. In this case the percent identity calculated by FASTDB is not manually corrected.

Of course, due to the degeneracy of the genetic code, one of ordinary skill in the art will immediately recognize that a large number of the nucleic acid molecules having a sequence at least 80%, 85%, 90%, 92%, 95%, 96%, 97%, 98% or 99% identical to the reference nucleic acid can encode a polypeptide that may bind an antigen, e.g., CD103. In fact, since degenerate variants of these nucleotide sequences all encode the same polypeptide, this will be clear to the skilled artisan even without performing the above described comparison assay.

The present invention is further directed to polynucleotides comprising, or alternatively consisting of, isolated nucleic acid molecules which encode domains of domains of the antibodies of the present invention.

In another aspect, the invention provides an isolated nucleic acid molecule comprising, or alternatively consisting of, a polynucleotide which hybridizes under stringent hybridization conditions to a portion of the polynucleotide in a nucleic acid molecule of the invention described above. By “stringent hybridization conditions” is intended overnight incubation at 42° C. in a solution comprising, or alternatively consisting of: 50% formamide, 5×SSC (750 mM NaCl, 75 mM trisodium citrate), 50 mM sodium phosphate (pH 7.6), 5×Denhardt's solution, 10% dextran sulfate, and 20 μg/ml denatured, sheared salmon sperm DNA, followed by washing the filters in 0.1×SSC at about 65° C.

By a polynucleotide which hybridizes to a “portion” of a polynucleotide is intended a polynucleotide (either DNA or RNA) hybridizing to at least about 15 nucleotides (nt), and more in particular at least about 20 nt, still more particular at least about 30 nt, and even more particular about 30-70 nt of the reference polynucleotide. In this context “about” includes the particularly recited value and values larger or smaller by several (5, 4, 3, 2, or 1) nucleotides. These polynucleotides may be useful as diagnostic probes and primers. By a portion of a polynucleotide of “at least 20 nt in length,” for example, is intended 20 or more contiguous nucleotides from the nucleotide sequence of the reference polynucleotide.

Of course, a polynucleotide which hybridizes only to a poly A sequence or to a complementary stretch of T (or U) resides, would not be included in a polynucleotide of the invention used to hybridize to a portion of a nucleic acid of the invention, since such a polynucleotide would hybridize to any nucleic acid molecule containing a poly (A) stretch or the complement thereof (i.e., practically any double-stranded cDNA generated from an oligo-dT primed cDNA library).

As indicated, nucleic acid molecules of the present invention which encode an antibody of the present invention may include, but are not limited to those encoding the amino acid sequence of the polypeptide, by itself; the coding sequence for the polypeptide and additional sequences, such as those encoding a leader or secretory sequence, such as a pre-, or pro- or prepro-protein sequence; the coding sequence of the polypeptide, with or without the aforementioned additional coding sequences, together with additional, non-coding sequences, including for example, but not limited to introns and non-coding 5′ and 3′ sequences, such as the transcribed, non-translated sequences that play a role in transcription, mRNA processing, including splicing and polyadenylation signals, e.g., ribosome binding and stability of mRNA; an additional coding sequence which codes for additional amino acids, such as those which provide additional functionalities. The sequence encoding the polypeptide may also be fused to a marker sequence, such as a sequence encoding a peptide which facilitates purification of the fused polypeptide. In certain embodiments, the marker amino acid sequence is a hexa-histidine peptide, such as the tag provided in a pQE vector (Qiagen, Inc.), as well as an HN-tag (alternating histidine and asparagine) among others, many of which are commercially available. As described in Gentz et al., Proc. Natl. Acad. Sci. USA 86:821-824 (1989), for instance, hexa-histidine provides for convenient purification of the fusion protein. The “HA” tag is another peptide useful for purification which corresponds to an epitope derived from the influenza hemagglutinin protein, which has been described by Wilson et al., Cell 37:767 (1984).

One fusion protein of the present invention may comprise a heterologous region from immunoglobulin that is useful to solubilize proteins. For example, fusion proteins comprising various portions of constant region of immunoglobin molecules together with another human protein or part thereof are well known in the art. In many cases, the Fc portion of the fusion protein is thoroughly advantageous for use in therapy and diagnosis and thus results, for example, in improved pharmacokinetic properties. On the other hand, for some uses it would be desirable to be able to delete the Fc part after the fusion protein has been expressed, detected and purified in the advantageous manner described. This is the case when Fc portion proves to be a hindrance to use in therapy and diagnosis, for example when the fusion protein is to be used as antigen for immunizations. In drug discovery, for example, human proteins, such as, hIL-5-receptor has been fused with Fc portions for the purpose of high-throughput screening assays to identify antagonists of hIL-5. See, D. Bennett et al., Journal of Molecular Recognition, Vol. 8:52-58 (1995) and K. Johanson et al., The Journal of Biological Chemistry, Vol. 270, No. 16:9459-9471 (1995).

The present invention further relates to variants of the nucleic acid molecules of the present invention, which encode portions, analogs or derivatives of the antibodies of the present invention. Variants may occur naturally, such as a natural allelic variant. By an “allelic variant” is intended one of several alternate forms of a gene occupying a given locus on a chromosome of an organism.

Non-naturally occurring variants may be produced using art-known mutagenesis techniques, which include, but are not limited to: oligonucleotide mediated mutagenesis, alanine scanning, PCR mutagenesis, site-directed mutagenesis (see, e.g., Carter et al., Nucl. Acids Res. 13:4331 (1986); and Zoller et al., Nucl. Acids Res. 10:6487 (1982)), cassette mutagenesis (see, e.g., Wells et al., Gene 34:315 (1985)), restriction selection mutagenesis (see, e.g., Wells et al., Philos. Trans. R. Soc. London Ser.A 317:415 (1986)).

Such variants include those produced by nucleotide substitutions, deletions or additions which may involve one or more nucleotides. The variants may be altered in coding regions, non-coding regions, or both. Alterations in the coding regions may produce conservative or non-conservative amino acid substitutions, deletions or additions. Especially preferred among these are silent substitutions, additions and deletions, which do not alter the properties and activities of the antibodies. Also especially preferred in this regard are conservative substitutions.

The present invention also relates to vectors which include the isolated DNA molecules of the present invention, recombinant host cells which are genetically engineered with the recombinant vectors, and the production of antibodies by recombinant techniques. To be sure “recombinant host cells” are cells that harbor heterologous nucleic acid, such as a recombinant vector, where as “host cell” are cells derived from the host subject. “Recombinant host cells” include “host cell” provided the cells derived from the host subject are harboring heterologous nucleic acid.

The polynucleotides may be joined to a vector containing a selectable marker for propagation in a host. Generally, a plasmid vector is introduced in a precipitate, such as a calcium phosphate precipitate, or in a complex with a charged lipid. If the vector is a virus, it may be packaged in vitro using an appropriate packaging cell line and then transduced into the recombinant host cells.

The DNA insert should be operatively linked to an appropriate promoter, such as the phage lambda PL promoter, the E. coli lac, trp and tac promoters, the SV40 early and late promoters and promoters of retroviral LTRs, to name a few. Other suitable promoters will be known to the skilled artisan. The expression constructs will further contain sites for transcription initiation, termination and, in the transcribed region, a ribosome binding site for translation. The coding portion of the mature transcripts expressed by the constructs will preferably include a translation initiating at the beginning and a termination codon (UAA, UGA or UAG) appropriately positioned at the end of the polypeptide to be translated.

As indicated, the expression vectors may include at least one selectable marker. Such markers include dihydrofolate reductase or neomycin resistance for eukaryotic cell culture and tetracycline or ampicillin resistance genes for culturing in E. coli and other bacteria. Representative examples of appropriate recombinant hosts cells include, but are not limited to, bacterial cells, such as E. coli, Streptomyces and Salmonella typhimurium cells; fungal cells, such as yeast cells; insect cells such as Drosophila S2 and Spodoptera Sf9 cells; animal cells such as CHO, COS and Bowes melanoma cells; and plant cells. Recombinant host cells also include host cells. Appropriate culture mediums and conditions for the above-described recombinant host cells are known in the art.

Other recombinant host cells include, but are not limited to cells derived from the graft or from the graft donor. For example, the methods of the present invention relate to introducing recombinant molecules into cells from the graft or donor. In one embodiment, the nucleic acids of the present invention are introduced into hematopoietic stem cells derived from the graft. The recombinant hosts are then reintroduced into the graft.

In addition to the use of expression vectors in the practice of the present invention, the present invention further includes novel expression vectors comprising operator and promoter elements operatively linked to nucleotide sequences encoding a protein of interest.

One example of such a vector is pHE4 which is described in detail below. Components of the pHE4-5 vector include: 1) a neomycinphosphotransferase gene as a selection marker, 2) an E. coli origin of replication, 3) a T5 phage promoter sequence, 4) two lac operator sequences, 5) a Shine-Delgarno sequence, 6) the lactose operon repressor gene (lacIq). The origin of replication (oriC) is derived from pUC19 (Invitrogen, Carlsbad, Calif.). The promoter sequence and operator sequences were made synthetically. Synthetic production of nucleic acid sequences is well known in the art. A nucleotide sequence encoding an antibody of the present invention is, for example, operatively linked to the promoter and operator by inserting the nucleotide sequence between the two restriction sites the pHE4 vector.

As noted above, the pHE4 vector contains a lacIq gene. LacIq is an allele of the ladI gene which confers tight regulation of the lac operator. The lacIq gene encodes a repressor protein which binds to lac operator sequences and blocks transcription of down-stream (i.e., 3′) sequences. The lacIq gene product, however, dissociates from the lac operator in the presence of either lactose or certain lactose analogs, e.g., isopropyl B-D-thiogalactopyranoside (IPTG). The antibody would thus not be produced in appreciable quantities in uninduced recombinant host cells containing the pHE4 vector. Induction of these recombinant host cells by the addition of an agent such as IPTG, however, would result in the expression of the antibody coding sequence.

The promoter/operator sequences of the pHE4 vector comprise a T5 phage promoter and two lac operator sequences. One operator is located 5′ to the transcriptional start site and the other is located 3′ to the same site. These operators, when present in combination with the lacIq gene product, confer tight repression of down-stream sequences in the absence of a lac operon inducer, e.g., IPTG. Expression of operatively linked sequences located down-stream from the lac operators may be induced by the addition of a lac operon inducer, such as IPTG. Binding of a lac inducer to the lacIq proteins results in their release from the lac operator sequences and the initiation of transcription of operatively linked sequences. Lac operon regulation of gene expression is reviewed in Devlin, T., Textbook of Biochemistry with Clinical Correlations, 4th Edition (1997), pages 802-807.

Among known bacterial promoters suitable for use in the production of proteins of the present invention include the E. coli lacI and lacZ promoters, the T3 and T7 promoters, the gpt promoter, the lambda PR and PL promoters and the trp promoter. Suitable eukaryotic promoters include the CMV immediate early promoter, the HSV thymidine kinase promoter, the early and late SV40 promoters, the promoters of retroviral LTRs, such as those of the Rous Sarcoma Virus (RSV), and metallothionein promoters, such as the mouse metallothionein-I promoter.

The pHE4 vectors also contain a Shine-Delgarno sequence 5′ to the AUG initiation codon. Shine-Delgarno sequences are short sequences generally located about 10 nucleotides up-stream (i.e., 5′) from the AUG initiation codon. These sequences essentially direct prokaryotic ribosomes to the AUG initiation codon.

Among vectors for use in bacteria include pQE70, pQE60 and pQE-9, available from Qiagen; pBS vectors, Phagescript vectors, Bluescript vectors, pNH8A, pNH16a, pNH18A, pNH46A, available from Stratagene; and ptrc99a, pKK223-3, pKK233-3, pDR540, pRIT5 available from Pharmacia. Among eukaryotic vectors are pWLNEO, pSV2CAT, pOG44, pXT1 and pSG available from Stratagene; and pSVK3, pBPV, pMSG and pSVL available from Pharmacia. Other suitable vectors will be readily apparent to the skilled artisan.

Introduction of the construct into the host cell can be effected by calcium phosphate transfection, DEAE-dextran mediated transfection, cationic lipid-mediated transfection, electroporation, transduction, infection or other methods. Such methods are described in many standard laboratory manuals, such as Davis et al., Basic Methods In Molecular Biology (1986).

The polypeptides may be recovered and purified from recombinant cell cultures by well-known methods including ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. In one embodiment, high performance liquid chromatography (“HPLC”) is employed for purification. Polypeptides of the present invention include naturally purified products, products of chemical synthetic procedures, and products produced by recombinant techniques from a prokaryotic or eukaryotic host, including, for example, bacterial, yeast, higher plant, insect and mammalian cells. Depending upon the host employed in a recombinant production procedure, the polypeptides of the present invention may be glycosylated or may be non-glycosylated. In addition, polypeptides of the invention may also include an initial modified methionine residue, in some cases as a result of host-mediated processes.

The present invention also relates to methods of detecting antigen in a biological sample. The antigen may be, but is not limited to CD103, CD8+, E-cadherin and TGF-β, or any portion thereof. Various methods can be used to detect the antigen in the sample, and can also be used to assess the functional activity of antibodies and fragments. In one embodiment, where one is assaying the ability to bind to or compete with a polypeptide for binding to, for example, CD103, various immunoassays known in the art can be used. The immunoassays include but are not limited to, competitive and non-competitive assay systems using techniques such as a planar array, a radioimmunoassays, ELISA (enzyme linked immunosorbent assay), “sandwich” immunoassays, immunoradiometric assays, gel diffusion precipitation reactions, immunodiffusion assays, in situ immunoassays (using colloidal gold, enzyme or radioisotope labels, for example), western blots, precipitation reactions, agglutination assays (e.g., gel agglutination assays, hemagglutination assays), complement fixation assays, immunofluorescence assays, protein A assays, and immunoelectrophoresis assays, etc. Specific embodiments of some of the assays listed include, but are not limited to, direct and indirect assays, as well as binary and tertiary sandwich assays. Other examples of assays that may be used in the methods of the present invention include, but are not limited to, bead or particle-based immunoassays, chemiluminescent assays, surface plasmon resonance (SPR) based assays, fluorescence assays, rolling-circle amplification assays, assays using dendrimers, and other enzyme or non-enzymatic amplification schemes.

The invention may also be used to screen antibodies or other compounds that have been developed as potential therapeutics, such as, but not limited to, humanized antibodies and antagonists to CD103 and/or TGF-β. Vaccination studies may be undertaken that have the intent of generating antibodies in the subject that bind and antagonize the effects of the antigen of the present invention. The antibodies of the present invention may be used to compare the affinity or other characteristics of generated antibodies or other molecules, such as candidate drugs or compositions that may bind a target molecule, such as, but not limited to, TGF-β, TGF-β receptor, CD103 and E-cadherin. Specifically, the invention provides methods of assessing the affinity of binding agents, e.g., antibodies, comprising competition assays between the antibodies of the present invention and a different binding agent.

In one embodiment, antibody or binding agent binding is detected by detecting a label on the antibody or primary binding agent. In another embodiment, the primary antibody or biding agent is detected by detecting binding of a secondary antibody or reagent to the primary antibody or primary binding agent. In a further embodiment, the secondary antibody is labeled. Many means are known in the art for detecting binding in an immunoassay and are within the scope of the present invention. In particular embodiments of the present invention, an antibody would be bound to a solid support (such as glass, polystyrene, polypropylene, polyethylene, dextran, nylon, amylases, natural and modified celluloses, polyacrylamides, agaroses, and magnetite). The nature of the carrier can be either soluble to some extent or insoluble for the purposes of the present invention. The support material may have virtually any possible structural configuration so long as the coupled molecule is capable of binding to the antigen. Thus, the support configuration may be spherical, as in a bead, or cylindrical, as in the inside surface of a test tube, or the external surface of a rod. Alternatively, the surface may be flat such as a sheet, test strip, etc. Those skilled in the art will note many other suitable carriers for binding antibody, or will be able to ascertain the same by use of routine experimentation.

For example, ELISA assays utilize a capture molecule that initially binds to the antigen is or can be bound to the wells of culture plate. In this embodiment, the culture plate is acting as the carrier for the binding agent, e.g., the antibody. Subsequently, a labeled detection molecule (which may recognize the capture molecule or the biomarker) may be added to the test environment. One of skill in the art would be knowledgeable as to the parameters that can be modified to increase the signal detected as well as other variations of ELISAs known in the art. As used herein the term “capture molecule” is used mean a binding agent that immobilizes the antigen by its binding to the antigen. Further, an antigen is “immobilized” if the antigen or antigen-capture molecule complex is separated or is capable of being separated from the remainder of the sample. When the capture molecule is coated to a well or other surface, a detection molecule may be added following the addition of the antigen of interest to the wells. As used herein, a detection molecule is used to mean a molecule, such as an antibody or receptor, comprising a label. In a specific embodiment, the methods of the present invention comprise the use of a capturing antibody and a detection antibody to detect the antigen. In a more specific embodiment, the capture antibody and the detection antibody are the same antibodies with the same binding specificities. In another specific embodiment, the capture antibody and the detection antibody are different antibodies.

A label, as used herein, is intended to mean a chemical compound or ion that possesses or comes to possess or is capable of generating a detectable signal. The labels may also provide other utility, such as, for example, providing a therapeutic benefit or increasing solubility or providing a means for isolating or purifying the antibodies or binding agents. The labels of the present invention may be conjugated to the primary binding agent, e.g., primary antibody, or secondary binding agent, e.g., secondary antibody, the antigen or a surface onto which the label and/or binding agent is attached. Examples of labels includes, but are not limited to, radiolabels, such as, for example, ³H and ³²P, that can be measured with radiation-counting devices; pigments, biotin, dyes or other chromogens that can be visually observed or measured with a spectrophotometer; spin labels that can be measured with a spin label analyzer; and fluorescent labels (fluorophores), where the output signal is generated by the excitation of a suitable molecular adduct and that can be visualized by excitation with light that is absorbed by the dye or can be measured with standard fluorometers or imaging systems. Examples of suitable radioisotopic labels include ¹¹¹In, ¹²⁵I, ¹³¹I, ³⁵S, ¹⁴C, ⁵¹Cr, ⁵⁷Co, ⁵⁸Co, ⁵⁹Fe, ⁷⁵Se, ¹⁵²Eu, ⁹⁰Y, ⁶⁷Cu, ²¹⁷Ci, ²¹¹At, ²¹²Pb, ⁴⁷Sc, ¹⁰⁹Pd etc. Examples of suitable non-radioactive isotopic labels include ¹⁵⁷Gd, ⁵⁵Mn, ¹⁶²Dy, ⁵²Tr, ⁵⁶Fe etc.

Additional examples of labels include, but are not limited to, a phosphorescent dye, a tandem dye and a particle. The label can be a chemiluminescent substance, where the output signal is generated by chemical modification of the signal compound; a metal-containing substance; or an enzyme, where there occurs an enzyme-dependent secondary generation of signal, such as the formation of a colored product from a colorless substrate. The term label also includes a “tag” or hapten that can bind selectively to a conjugated molecule such that the conjugated molecule, when added subsequently along with a substrate, is used to generate a detectable signal. For example, one can use biotin as a label and subsequently use an avidin or streptavidin conjugate of horseradish peroxidate (HRP) to bind to the biotin label, and then use a colorimetric substrate (e.g., tetramethylbenzidine (TMB)) or a fluorogenic substrate such as Amplex Red reagent (Molecular Probes, Inc.) to detect the presence of HRP. Numerous labels are know by those of skill in the art and include, but are not limited to, particles, fluorophores, haptens, enzymes and their colorimetric, fluorogenic and chemiluminescent substrates and other labels that are described in RICHARD P. HAUGLAND, MOLECULAR PROBES HANDBOOK OF FLUORESCENT PROBES AND RESEARCH PRODUCTS (9^{th} edition, CD-ROM, (September 2002), which is herein incorporated by reference.

The antibodies or binding agents of the present invention may also be conjugated to additional compounds or proteins that may not provide a signal. In one embodiment, the antibodies or binding agents may be conjugated for the purposes of selectively delivering a therapeutic compound to cells. For example, antibodies to CD103 may be generated using the methods of the present invention, and these antibodies may be labeled with a radioactive label, such that the antibody is capable of delivering radioactivity to cells expressing CD103. Examples of radioactivity that may be delivered to cells expressing, for example CD103, E-cadherin, CD8 or TGF-β receptor, include but are not limited to ³²P, ³³P, ⁴¹Sc, ⁶⁴Cu, ⁶⁷Cu, ⁷⁷As, ⁹⁰Y, ¹⁰⁵Ph, ¹⁰⁹Pd, ¹¹¹Ag, ¹²⁵I, ¹⁴³Pr, ¹⁵³Sm, ¹⁶¹Tb, ¹⁶⁶Ho, ¹⁷⁷Lu, ¹⁸⁶Re, ¹⁸⁸Re, ¹⁸⁹Re, ¹⁹⁴Ir, ¹⁹⁹Au, ²¹²Pb, and ²¹³Bi.

The antibodies or binding agents may be conjugated with other therapeutic agents for “target-directed therapy” for treating specific populations of cells within either the graft or the host, or both. Methods of using antibodies for target-directed therapies are disclosed in U.S. Pat. No. 7,011,812, which is incorporated by reference. In one embodiment, the antibodies or binding agents further comprise a toxin such that the toxin is delivered to, for example, the CD103+ cells of a graft. Examples of toxins include, but are not limited to saporin and ricin. The conjugated moieties may also provide other utility, such as, for example, providing a therapeutic benefit or increasing solubility or providing a means for isolating or purifying the antibodies or binding agents.

The present invention also relates to methods of screening a graft to determine the likelihood that the graft will generate graft versus host disease (GVHD) after transplantation. The methods of screening comprise determining the levels of CD103 in the graft prior to transplantation. The determined levels of CD103 in the graft can then be used to assess the likelihood that the host will develop GVHD after transplantation of the graft.

In one embodiment, a sample would be taken from the graft and would be placed in contact with a bound antibody that is specific for CD103, under conditions sufficient to permit CD103, which may be present in the sample, to bind to the support-bound antibody. In one specific embodiment, the support could then be incubated in the presence of a labeled antibody that is specific for CD103 under conditions sufficient to permit the labeled antibody to bind to an open binding site on CD103. After washing away unbound molecules of the labeled antibody, the amount of label is then determined. The presence labeled antibody bound to the support indicates the presence of CD103 in the sample.

As will be readily perceived, any of a large number of equivalent assays may be alternatively employed without departing from the spirit of the above-described assay. For example, the assay can be conducted in liquid phase rather than through the use of a solid support. Other variations of immunoassays can be also employed.

The methods described herein can be used to measure of the levels for the purposes of the present invention. The measurement of the levels of biomarker may be qualitative or quantitative. For example, the levels of biomarker may be quantified is some numerical expression, such as a ratio or a percentage.

Once the levels of CD103 have been determined, these levels are used to determine the likelihood that the host will develop GVHD in response to transplantation of the graft. To assess the likelihood of the host developing GVHD, the determined levels can be compared to previously determined levels of CD103 where the hosts developed GVHD after transplantation. The determined levels may be compared to a single data point, or it may be compared to a population of data. The predetermined levels of CD103 may be categorized or further correlated according to sex, age, weight, ethnicity, geographic location, fasting state, state of pregnancy or post-pregnancy, menstrual cycle, general health of the subject, alcohol or drug consumption, caffeine or nicotine intake and circadian rhythms.

The present invention also relates to methods of screening compounds to treat graft versus host disease (GVHD), with the methods comprising administering a candidate compound to CD103; and determining the level of binding between said candidate and CD103. Determining levels of biding between candidate compounds and CD103 are described herein. In one embodiment, the CD103 to which the candidate compound is administered is in a cell-free environment. In another embodiment, the CD103 to which the candidate compound is administered is expressed on a cell.

The present invention also provides pharmaceutical compositions. Such compositions comprise a therapeutically effective amount of a compound, and a pharmaceutically acceptable carrier. In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. Further, a “pharmaceutically acceptable carrier” will generally be a non-toxic solid, semisolid or liquid filler, diluent, encapsulating material or formulation auxiliary of any type.

The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is may be the carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents such as acetates, citrates or phosphates. Antibacterial agents such as benzyl alcohol or methyl parabens; antioxidants such as ascorbic acid or sodium bisulfite; chelating agents such as ethylenediaminetetraacetic acid; and agents for the adjustment of tonicity such as sodium chloride or dextrose are also envisioned. These compositions can take the form of solutions, suspensions, emulsion, tablets, pills, capsules, powders, sustained-release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in Remington's Pharmaceutical Sciences by E. W. Martin, which is incorporated by reference. Such compositions will contain a therapeutically effective amount of the compound, together with a suitable amount of carrier so as to provide the form for proper administration to the patient. The formulation should suit the mode of administration. The parental preparation can be enclosed in ampoules, disposable syringes or multiple dose vials made of glass or plastic.

The composition may be formulated in accordance with routine procedures as a pharmaceutical composition adapted for intravenous administration to human beings. Typically, compositions for intravenous administration are solutions in sterile isotonic aqueous buffer. Where necessary, the composition may also include a solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The compounds of the invention can be formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

The amount of the compound of the invention which will be effective in the treatment, inhibition and prevention of GVHD may be determined by standard clinical techniques. In addition, in vitro assays of the present invention may optionally be employed to help identify optimal dosage ranges. The precise dose to be employed in the formulation will also depend on the route of administration, and the seriousness of the disease or disorder, and should be decided according to the judgment of the practitioner and each patient's circumstances. Effective doses may be extrapolated from dose-response curves derived from in vitro or animal model test systems.

As a general proposition, the total pharmaceutically effective amount of composition administered parenterally per dose will be in the range of about 1 μg/kg/day to 10 mg/kg/day of patient body weight, although, as noted above, this will be subject to therapeutic discretion. In particular, this dose is at least 0.01 mg/kg/day, and more particularly, for humans, between about 0.01 and 1 mg/kg/day for the hormone. If given continuously, the composition may typically be administered at a dose rate of about 1 μg/kg/hour to about 50 μg/kg/hour, either by 1-4 injections per day or by continuous subcutaneous infusions, for example, using a mini-pump. An intravenous bag solution may also be employed.

For antibodies, the dosage administered to a patient is typically 0.1 mg/kg to 100 mg/kg of the patient's body weight. Preferably, the dosage administered to a patient is between 0.1 mg/kg and 20 mg/kg of the patient's body weight, more preferably 1 mg/kg to 10 mg/kg of the patient's body weight. Generally, human antibodies have a longer half-life within the human body than antibodies from other species due to the immune response to the foreign polypeptides. Thus, lower dosages of human antibodies and less frequent administration is often possible. Further, the dosage and frequency of administration of antibodies of the invention may be reduced by enhancing uptake and tissue penetration, e.g., into the brain, of the antibodies by modifications such as, for example, lipidation.

The invention also provides a pharmaceutical pack or kit comprising one or more containers filled with one or more of the ingredients of the pharmaceutical compositions of the invention, and, optionally instructions or product insert.

The following examples are meant to illustrate certain embodiments of the present invention and should not be taken as any limit to the scope of the claimed invention.

### EXAMPLES

### Methods and Materials for Examples

Animals—2C T cell receptor transgenic (TCR-tg) mice (H-2^{b}) which express a T cell receptor with specificity towards an 8-mer peptide (p2Ca) Sha, W. C., et al., Nature 335:271-274 (1988); Udaka, K., et al., Proc Natl Acad Sci USA 90:11272-11276 (1993)) were originally obtained from Dr. Ted Hansen (Washington University School of Medicine). The 2C transgenic mice were maintained by backcrossing heterozygous 2C males to C57BL/6 females and screening the offspring for expression of the clonotypic 2C TCR by FACS of peripheral blood using the 1B2 monoclonal antibody (mAb). Mice expressing a dominant negative type II TGF-β receptor (DNR) (Lucas, P. J., et al., J Exp Med 191:1187-1196 (2000)), as well as 2C mice that were bred onto the DNR background (2C-DNR) (NIH, Bethesda, Md., USA) were used in some experiments. C57BL/6 (B6) (H-2b) and BALB/c (H-2^{d}) mice were purchased from Jackson Laboratory (Bar Harbor, Me., USA). 2C CD103−/− mice were generated by breeding the 2C(H-2b) mice and CD103−/−C57BL/6 (H-2^{b}) mice (Schon, M. P., et al., Immunol 162:6641-6649 (1999)), followed by backcrossing the 2C CD103^{+/-} F1 generation to the CD103^{-/-} parental strain to generate 2C CD103^{-/-} mice. CD103^{-/-} mice were identified by surface staining for the TCR transgene and polymerase chain reaction (PCR) for the homozygous CD103^{-/-} mutation. Wild type congenic 2C Thy1.1⁺ mice were obtained by interbreeding 2C Thy1.2⁺/Thy1.2⁺ and C57BL/6 Thy1.1⁺/Thy1.1⁺ animals to generate a 2C Thy1.⁺/Thy1.2⁺ F1 generation. All mice were maintained under specific pathogen-free conditions in the animal facility at the University of Maryland, Baltimore. All animal studies were approved by the Institutional Review Board.

Production of Antibodies Specific for CD103—1B2 mAb (anti-clonotypic 2C TCR; mouse IgG1) (Kranz, D. M., et al., Proc Natl Acad Sci USA 81:573-577 (1984)) was obtained from hybridoma culture supernatant. Biotinylated rat anti-mouse IgG1 was purchased from Caltag (Burlingame, Calif., USA). Directly conjugated anti-mouse antibodies used included CD8-FITC, CD8-PerCP, Thy1.1-PerCP, Thy1.2-PE, CD44-FITC, CD44-PE, CD62L-PE, M290 (anti-CD103)-PE, CD11a-PE, CD25-PE, IFN-γ-PE, TNF-α-PE and the species- and isotype-matched controls, all of which were purchased from BD PharMingen (San Diego, Calif. USA).

Adoptive Transfer and Bone Marrow Transplantation—Lethally irradiated (8 Gy, ¹³⁷Cs source) BALB/c (H-2^{d}) mice were reconstituted within 4-6 hours by a single 0.5 ml intravenous inoculum containing 10×10⁶ B6 bone marrow cells (BMC) and 10×10⁶ B6 spleen cells (SC) along with either 1×10⁶ 2C SC or 1×10⁶ 2C-DNR cells. In some experiments, equal numbers (0.5×10⁶) of wild type Thy1.1+2C and Thy1.1⁺2C-DNR spleen cells were mixed together and adoptively transferred in combination with B6 BMC and SC into lethally-irradiated BALB/c hosts.

In adoptive transfer experiments intended to elucidate the role of CD103 in lymphocyte migration to the intestine, equal numbers (0.5×10⁶) of wild type Thy1.1⁺ 2C spleen cells and CD103^{-/-} Thy1.1⁻ 2C spleen cells were mixed and adoptively transferred into a group of lethally irradiated BALB/c hosts. In some experiments, 1×10⁶ wild type or CD103^{-/-} 2C cells were separately transferred into lethally irradiated BALB/c mice in combination with C57BL/6 BM and SC.

To examine the survival and organ pathology in recipients of CD103^{-/-} CD8 cells, two groups of lethally irradiated BALB/c hosts received 10×10⁶ B6 BMC and either 10×10⁶ CD8-enriched B6 SC or 10×10⁶ CD8-enriched B6 CD103⁻⁻ SC. In these experiments, donor spleen cells were enriched for naive CD8⁺ T cells by treatment with a mAb to CD4 (RL172.4), followed by incubation in 1/10 Low-Tox M rabbit complement (Accurate Chemical and Scientific, Westbury, N.Y., USA). The resulting cell suspensions contained <0.5% CD4⁺ T cells.

To ensure that equivalent numbers of CD8 cells were transferred in different experiments, flow cytometry analysis using anti-CD8 and TCR-αβ was performed on each preparation to assess the degree of enrichment for CD8⁺ T cells. Additional experiments were performed by transferring 10×10⁶ B6 BMC alone. Mice were randomly grouped before and after irradiation and were given acidified drinking water to prevent infection. Control irradiated untreated mice were also included in each experiment.

Lymphocyte Isolation—Lymphocytes infiltrating the intestinal epithelium during GVHD were isolated as previously described in Guy-Grand, D., et al., J Exp Med 148:1661-1677 (1978), with modifications. The small intestine was flushed with PBS. After removal of the Peyer's patches and fat, the intestine was divided longitudinally and cut into 2-mm sections. The mucosa was scraped and dissociated by mechanical disruption on a stirring platform for 15 min in RPMI 1640 containing 10% FCS and 1 mM dithioerythritol. Tissue debris and cell aggregates were removed by passage over a glass wool column in RPMI 1640-10% FCS. The lymphocytes were obtained by centrifugation on Lympholyte-M (Cedarlane Laboratories Limited, Hornby, Ontario, CANADA) and the cells were suspended in complete medium.

Lymphocytes were isolated from the kidney as previously described in Wang, D., et al., J Immunol 172:214-221 (2004), incorporated by reference. Briefly, the kidney was minced and the resulting fragments incubated for 30 min in medium containing collagenase (Worthington Biochemical, Freehold, N.J., USA), soybean trypsin inhibitor (Sigma-Aldrich, St. Louis, Mo., USA), and DNase (Roche, Indianapolis, Ind., USA). Lymphocytes were isolated by centrifugation on Lympholyte-M (Cedarlane Laboratories). Cells were isolated from the mesenteric lymph nodes (MLN) and spleen of recipients by mincing with forceps and passage of the resulting cell suspension through nylon mesh of 100-μm pore size. Lung- and liver-infiltrating lymphocytes were harvested as described in Masopust, D., et al., Science 291:2413-2417 (2001), but with modifications. Following perfusion with phosphate-buffered saline (PBS), liver and lung tissue was minced and incubated with stirring at 37° C. for 30 min in medium containing collagenase (Worthington Biochemical), soybean trypsin inhibitor (Sigma-Aldrich), and DNase (Roche). Lymphocytes were isolated by centrifugation on Lympholyte-M (Cedarlane Laboratories). Lymphocyte populations were washed twice in FACS buffer prior to antibody staining and FACS analyses.

Histology—Host organs were harvested at the designated times, fixed in 10% buffered formalin, and embedded in paraffin. Sections (6 μm) were stained with H&E and blindly analyzed.

Flow Cytometry Analysis—For three-color flow cytometry analysis, harvested cells were stained with PerCP-conjugated mAb to CD8b in combination with FITC- and PE-conjugated mAbs to markers of interest. For four-color flow cytometry analysis, cells were stained with anti-CD8a-FITC and anti-Thy1.1-PerCP in combination with PE- and APC-conjugated antibodies. Species and isotype-matched mAbs of irrelevant specificity were used as controls for non-specific fluorescence. After staining, cells were fixed with 0.5% paraformaldehyde and analyzed using a FACSCalibur (Becton Dickinson, San Jose, Calif., USA).

For intracellular cytokine staining (ICS), harvested cells were activated with PMA and ionomycin, followed by addition of 4 μl/ml of Monensin (Golgi Stop, Pharmingen) for an additional 6 hours at 37° C. Cells were washed and resuspended in staining buffer followed by surface staining. Cells were then washed and resuspended in Cytoperm/Cytofix solution (PharMingen) for 20 min on ice and subsequently incubated on ice for 30-60 min with appropriate dilutions of anti-cytokine or isotype control antibodies. For Annexin-V staining, lymphocytes were stained in FACS® buffer at 4° C. with FITC-conjugated mAb to CD8b and APC-conjugated mAb to Thy1.1. Cells were then washed and incubated in Annexin-V binding buffer with PE-Annexin-V at 1:20 dilution and 7AAD (to exclude non-viable cells) for 15 min at room temperature. The cells were washed, resuspended in Annexin-V binding buffer and analyzed within 1 hour.

Lymphocyte populations were gated by forward scatter/side scatter analysis to exclude monocytes and debris. WinMDI 2.8 software developed by Dr. Joseph Trotter (Scripps Institute, San Diego, Calif.) (downloaded from the internet at facs.scripps.edu/software.html) was used for analysis and graphic display of flow cytometry data. The percentage of positive cells for a given marker was based on cutoff points chosen to exclude >99% of the negative control population.

Statistical Analyses—Data are expressed as mean ±SEM. To assess statistical significance, Student's t tests were performed using SigmaPlot 2000 software (SPSS Inc., Chicago, Ill., USA). P values of less than 0.05 were considered statistically significant.

### Example 1

### Assessing the Role of CD103 in Graft Versus Host Disease

Gut-specific Expression of CD103 by hsCD8eff—To determine if (hsCD8eff) populations express CD103, lethally irradiated BALB/c (L^{d+}) mice received 10×10⁶ BMC and 10×10⁶ SC from C57BL/6 (B6) (H2^{b}) donors plus trace numbers of spleen cells from 2C TCR-transgenic (B6) mice. Peripheral CD8+ cells from 2C mice express a TCR with specificity for H-2L^{d}, which is readily detected using a mAb (1B2) directed to the clonotypic 2C TCR. Thus, use of mAbs to CD8 and 1B2 in multi-color FACS analyses allowed the phenotypic attributes of donor CD8 effector populations targeting host L^{d} (host-specific; CD8+1B2+ cells) to be monitored in vivo during the course of GVHD.

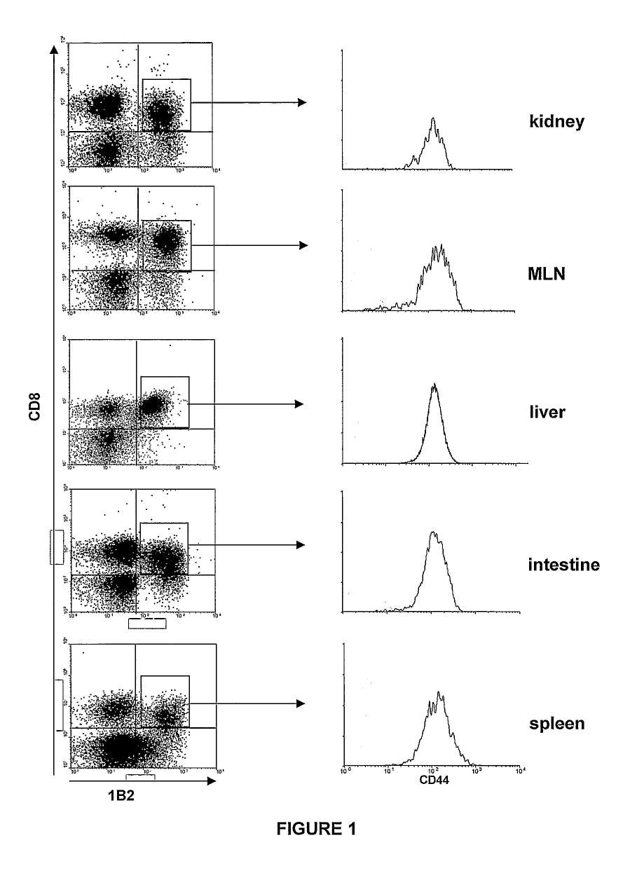

Prior to transfer, the vast majority of host-specific CD8⁺ T cells (1B2⁺CD8⁺) contained within the inoculum were CD44⁻CD11a^{lo}CD62L^{hi} indicating a naive phenotype (data not shown). FIG. 1 shows that, following adoptive transfer into lethally irradiated recipients, 1B2⁺CD8⁺ cells infiltrated host lymphoid (spleen and MLN) and non-lymphoid (kidney, liver and intestine) compartments as early as day 5 post-BMT and were readily detected by day 7. All such cells displayed an effector phenotype (CD44^{hi}CD11a^{hi}CD62L^{lo}). Migration of donor effector T cells into peripheral compartments correlated with the development of classic manifestations of GVHD, e.g., diarrhea, weight loss, wasting and mortality (MST=7.5±0.3 days; range=6-21 days).

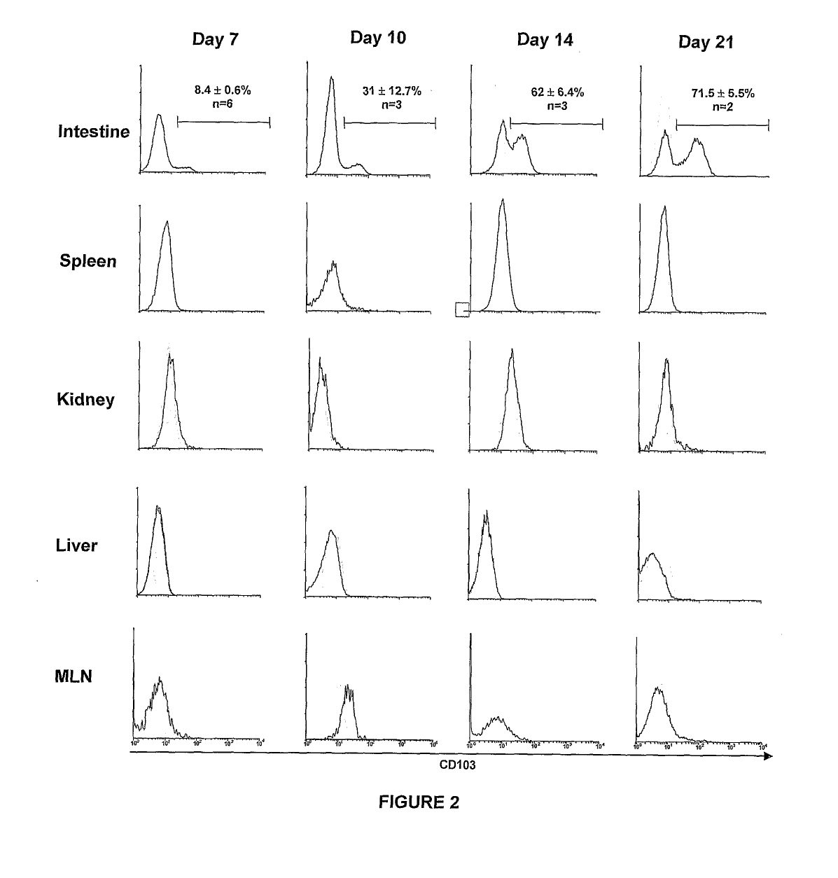

As shown in FIG. 2, a major subset of hsCD8eff that infiltrated the host intestinal epithelium during GVHD expressed significant levels of CD103, the percentage of which progressively increased with time. Thus, at day 7 post-BMT, 8.4±0.6% (n=6) of the IB2⁺CD8⁺ T cells expressed CD103, and this fraction increased to 31±12.7% (n=3) by day 10, 62±6.4% (n=3) by day 15, and 71.5±5.5% (n=2) by day 21. FIG. 2 also demonstrates that hsCD8eff cells that infiltrated other host compartments, including the spleen, kidney, liver, and MLN, expressed negligible levels of CD103 at all time points examined. The mean percentage ±SE of 1B2⁺CD8⁺ T cells in the spleen which expressed significant levels of CD103 was 0.36±0.2, 1.2±0.87, 2.57±1.1, and 0.8±0.2 at days 7, 10, 14, and 21, respectively (all values P<0.05 compared to the intestine).

Non-transgenic CD8 effectors (1B2⁻CD8⁺CD44^{hi} cells) that infiltrated the host intestine also selectively expressed CD103, demonstrating that gut-specific expression of CD103 is not unique to TCR-transgenic CD8 cells (data not shown). Thus, these data revealed that CD103 is selectively expressed by hsCD8eff infiltrating the intestinal epithelium during the course of GVHD.

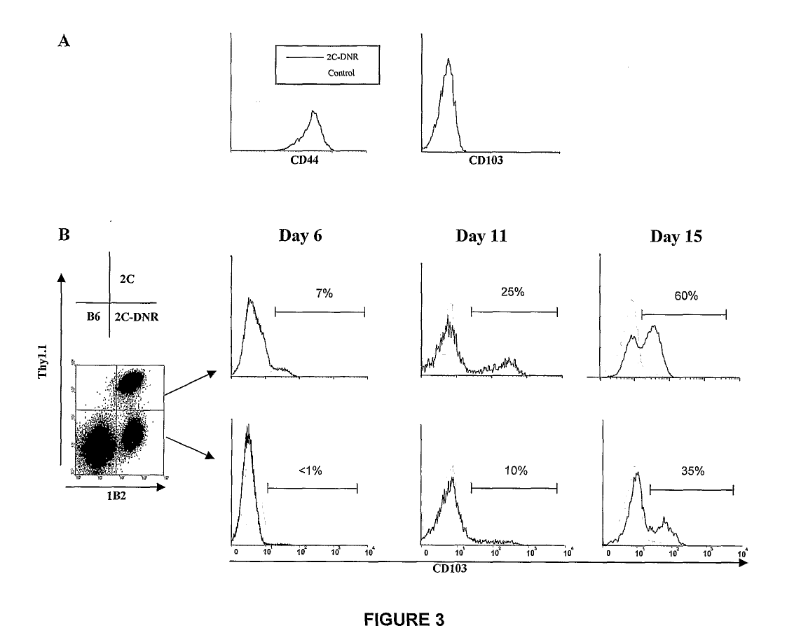

TGF-β Dependent Expression of CD103 in Gut-Derived hsCD8eff—2C TCR-transgenic mice expressing a dominant negative TGF-β type II receptor (2C-DNR) were used to determine if TGF-β activity regulates CD103 expression by gut-infiltrating hsCD8eff cells. The substitution of 2C-DNR cells for wild type 2C cells allowed an assessment of the contribution of TGF-β signaling to induction of CD103 expression by hsCD8eff infiltrating the intestinal epithelium. In these experiments, lethally-irradiated BALB/c recipients were adoptively transferred with B6 BM and SC along with 2C-DNR cells. As shown in FIG. 3A, and similar to wild type 2C, 2C-DNR cells migrated into the intestinal epithelium by day 7 post-BMT, and displayed an effector phenotype as indicated by upregulation of CD44. In contrast to their wild type counterparts, however, 2C-DNR cells that migrated into the intestinal epithelium were completely devoid of CD103 expression.

To directly compare CD103 expression by wild type versus 2C-DNR cells in the same mouse, equal numbers (0.5×10⁶) of each cell population were transferred together into lethally irradiated BALB/c recipients. To discriminate between the two populations of TCR-transgenic cells, wild type 2C cells expressing the congenic marker, Thy1.1, were generated, thus allowing identification of wild type 2C (1B2⁺CD8⁺Thy1.1⁺) and 2C-DNR (1B²+CD8⁺ Thy1.1⁻) cells using four-color FACS® analyses. As shown in FIG. 3B (top), a progressively increasing subset of wild type 2C cells infiltrating the intestinal epithelium were able to upregulate CD103. In contrast, a significantly lower percentage of 2C-DNR cells expressed CD103 at the same site at all time points examined (FIG. 3B (bottom)). At day 15 post-BMT, the mean percentage (±SE) of CD103-expressing cells among wild type 2C was 43.6±12.4 (n=3) as compared to 22.2±8.7 (n=3) for 2C-DNR cells (P=0.01). Thus, these data support an important role for TGF-β activity in regulating CD103 expression by hsCD8eff that infiltrate the host intestinal epithelium.

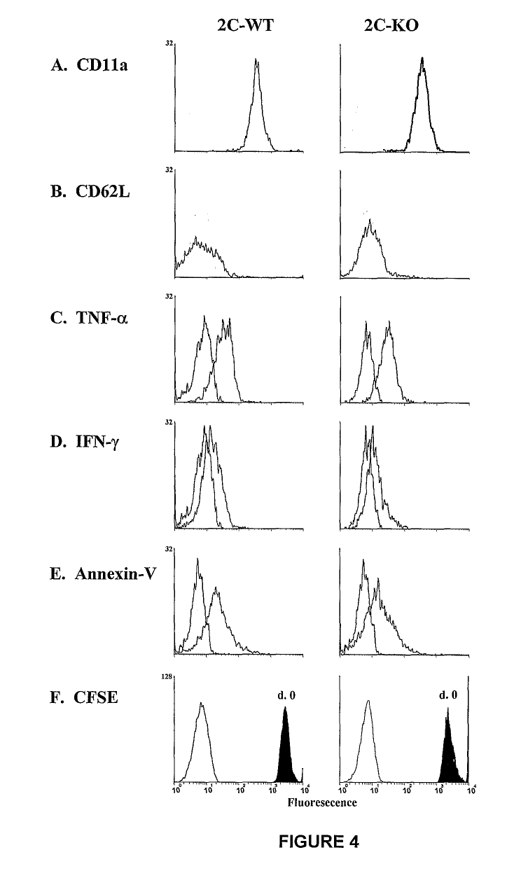

CD103 Expression Promotes Retention of hsCD8eff in the Intestinal Epithelium—To determine if CD103 expression promotes retention of CD103⁺CD8⁺ effectors in the host intestinal epithelium during GVHD, via E-cadherin, 2C mice were crossed onto the CD103^{-/-} background. The resulting CD103^{-/-} 2C effectors were compared with wild type counterparts for their capacity to migrate into the host intestinal epithelium. In these experiments, equal numbers (0.5×10⁶) of wild type 2C (Thy1.1⁺1B2⁺) cells and CD103^{-/-} 2C (Thy1.1⁻1B2⁺) cells, combined with B6 BM and SC, were transferred into lethally irradiated BALB/c hosts (Thy1.2⁺/Thy1.2⁺). Host organs were harvested at day 6, 12, 21, and 28 post-transfer, and the infiltrating lymphocytes were subjected to four-color FACS® analyses. Prior to transfer CD8⁺ T cells derived from both donor populations displayed a naive phenotype (data not shown), but following transfer both populations rapidly acquired a CD62L^{lo}CD11a^{hi} phenotype characteristic of T effector cells (FIGS. 4A and 4B). Furthermore, wild type and CD103^{-/-} hsCD8eff secreted comparable levels of the effector cytokines TNF-α (FIG. 4C) and IFN-γ (FIG. 4D).

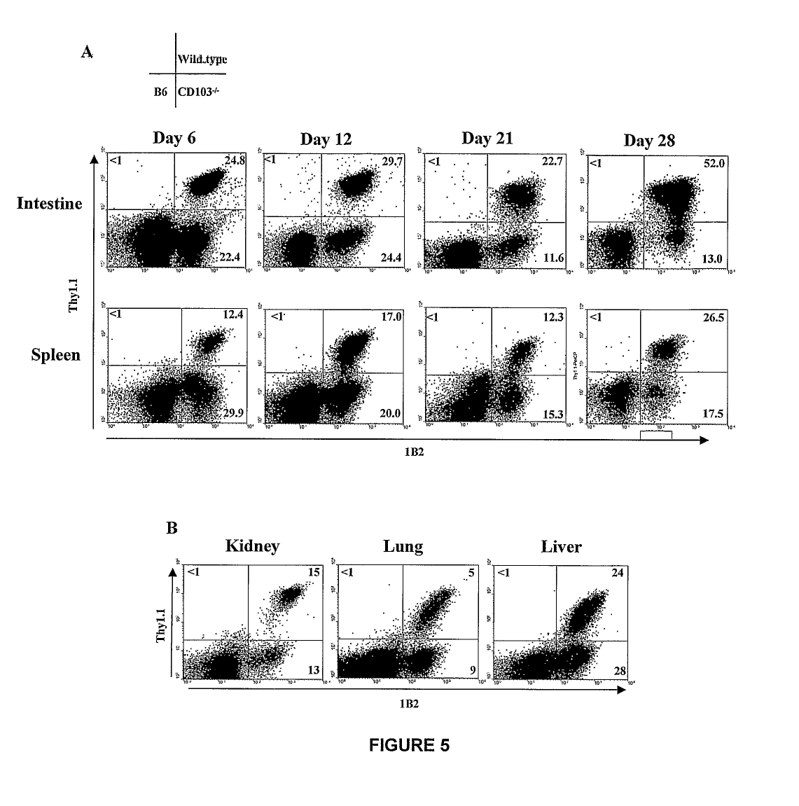

As shown in FIG. 5A (top), CD103^{-/-} hsCD8eff comprised 22% of the total CD8 T cells infiltrating the host intestinal epithelium at day 6 post-transfer. This proportion of hsCD8eff cells was comparable to that of the wild type hsCD8eff (FIG. 5A), indicating that CD103 expression is not required for initial migration of hsCD8eff into the intestinal epithelium. The numbers of wild type hsCD8eff cells, however, increased with time when compared to their CD103^{-/-} counterparts at later time points; i.e., the ratio of wild type: CD103^{-/-} hsCD8eff increased to 2:1 by day 21 and 4:1 by day 28. Selective retention of wild type vs. CD103^{-/-} hsCD8eff was also manifest in the spleen by day 28 post-transfer (FIG. 5A) but not in any of the non-lymphoid organs examined including the kidney, lung, and liver (FIG. 5B).

CD103 Expression Promotes CD8⁺ T-Cell Mediated Destruction Host Intestinal Epithelium—Hosts transferred with wild type 2C cells experienced more rapid mortality (MST=7.5±0.3 days, n=4) than those receiving CD103^{-/-} 2C cells (MST=24.7±5.9 days, n=4) or 2C-DNR cells (MST=16.3±2.4 days, n=4) (data not shown). These data suggested that CD103 expression by hsCD8eff is causally related to the development of GVHD pathology and mortality.

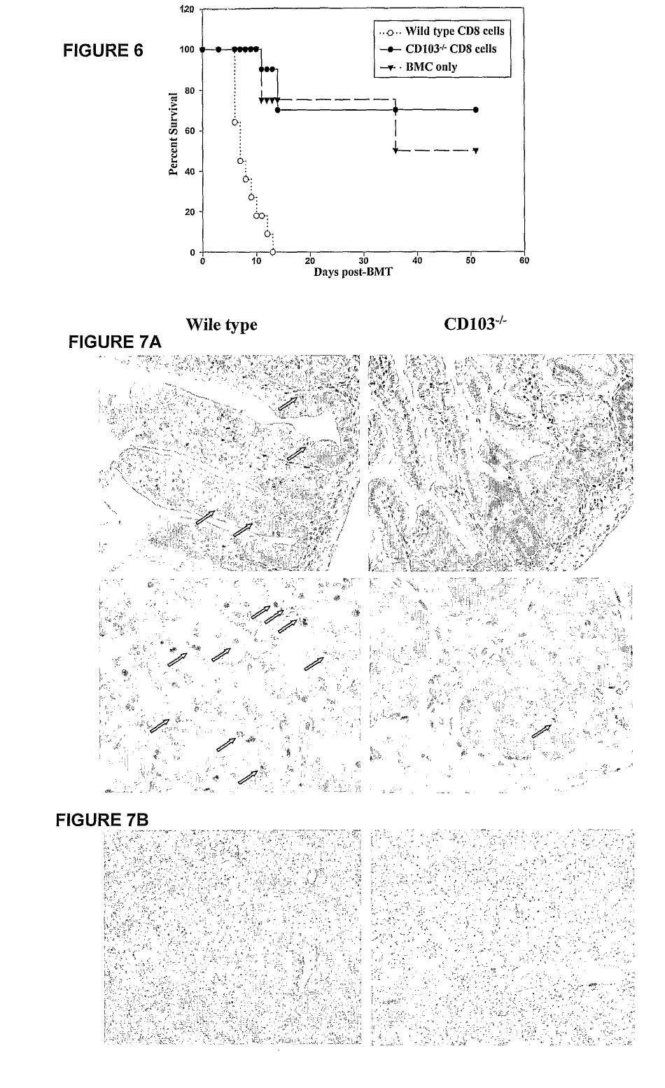

To determine the impact of CD103 expression on GVHD mediated by polyclonal CD8⁺ T cells, two groups of BALB/c mice were lethally irradiated and adoptively transferred with CD8-enriched spleen cells (<0.5% CD4 contamination) obtained from either wild type or CD103−/− donors (polyclonal B6 background) in combination with B6 BM. An additional control group received B6 BMC alone. Hosts from each group were monitored for survival, and intestinal specimens were analyzed histologically. As shown in FIG. 6, recipients of CD103^{-/-} CD8-enriched T cells enjoyed dramatically prolonged survival times (MST>48.6±6.5 days, n=10) compared to recipients of wild type cells (MST=8.2±0.8 days, n=11) (P<0.001). Indeed, recipients of CD103^{-/-} CD8 cells exhibited mortality kinetics similar to recipients of BMC alone.

As shown in FIG. 7A, histopathologic evaluation of intestinal specimens from mice transferred with wild type cells revealed more pronounced destruction than that seen in cohorts receiving CD103^{-/-} cells; e.g., villus atrophy, architectural distortion, lymphocytic infiltration, and appearance of apoptotic bodies. Quantitative analysis of the latter in the two groups revealed a four-fold increase in the number of apoptotic bodies in recipients of wild type cells compared to recipients of CD103^{-/-} cells. Analyses of liver pathology revealed a mild to moderate cellular infiltrate in both groups (FIG. 6C).

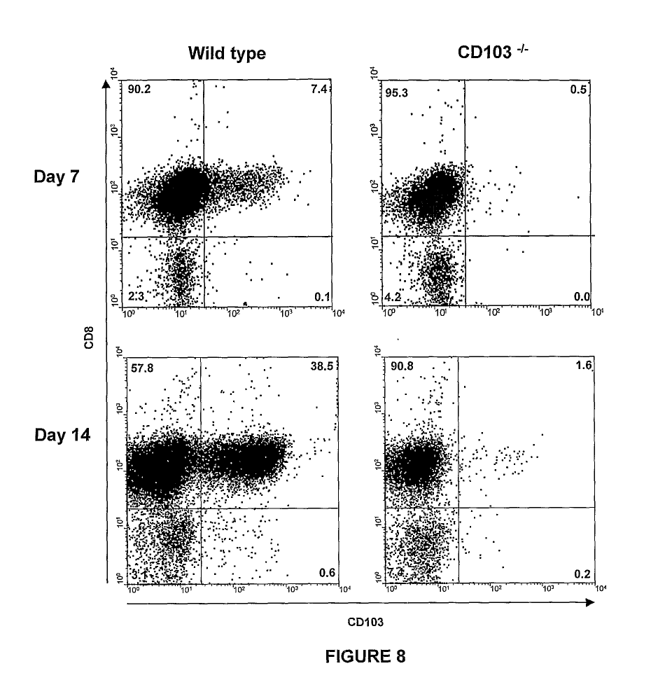

As shown in FIG. 8, FACS® analyses of donor-origin lymphocytes infiltrating the intestinal epithelium at day 14 post-BMT in recipients of wild type lymphocytes revealed a vast predominance of CD8 effectors (80-90% of infiltrating lymphocytes), ˜40% of which expressed CD103. Note that CD103 expression by gut-infiltrating lymphocytes was almost entirely confined to the CD8 subset (FIG. 8). There was a corresponding two-fold decrease in the abundance of CD103^{-/-} cells in the intestinal epithelium as compared to wild type counterparts (not shown). Thus, these data are consistent with a key role for CD103 in promoting both retention and pathogenic potential of polyclonal host-reactive CD8 effectors that infiltrate the host intestinal epithelium during GVHD.

### Example 2

### Separability of GVHD vs. GVT Effects Mediated by CD8⁺ T Cells

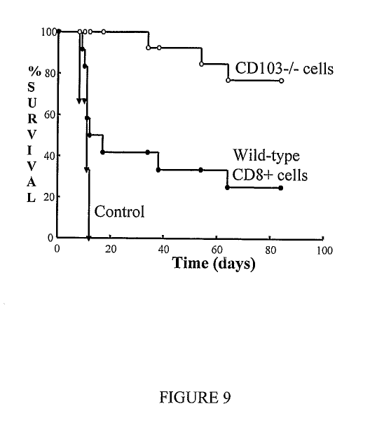

Bone marrow cells and purified CD8+ T cells from CD103−/− or wild type (WT) BALB/cJ (H-2d) mice were adoptively transferred into lethally-irradiated A/J (H-2a) mice together with the A/J fibroblast sarcoma (SaI/N). Incidence of GVHD and GVT was observed and target organs were assayed by histology and flow cytometry. Donors were primed with 1×107 A/J SC i.p. at day −5. FIG. 9 shows that CD8+ T cells from wild type BALB/cJ mice previously primed to A/J alloantigens readily transferred severe gut and liver GVHD (75% mortality at 3 months) (closed circles), and greatly inhibited tumor growth (17-fold reduction of mean tumor volume vs. control) (data not shown). In contrast, primed CD8+ T cells from CD103−/− mice exhibited only mild to undetectable GVHD (23% mortality at 3 months) (open circles), but still efficiently inhibited tumor growth (20-fold reduction vs. control) (data not shown).

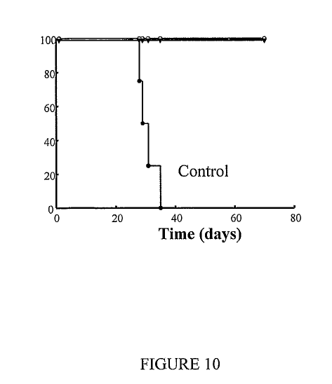

In a separate experiment, FIG. 10 shows that CD103−/− and wild-type CD8+ T cells both efficiently eliminated SaI/N tumors transplanted into syngeneic BALB/SCID mice with no tumor metastasis detected in either group at 10 weeks, thereby excluding the possibility that GVHD may contribute to tumor elimination. CD8+ T cells from wild-type (open circles) or CD103−/− (triangles) donors primed A/J alloantogens were adoptively transferred into histocompatible BALB/SCID mice together with the A/J fibrosarcoma SaI/N. A control group received SaI/N tumor only (closed circles). CD103−/− and WT CD8 T cells both efficiently eliminated SaI/N tumors transplanted into syngeneic BALB/scid mice, with no tumor metastasis detected in either group at 10 weeks. These data demonstrate that blocking CD103 activity in cells originating from the graft can abrogate GVHD while sparing beneficial GVT effects mediated by CD8+ T cells.

### Example 3

### Selective Depletion of CD8+ T cells in Bone Marrow

To test the ability to selectively destroy CD8+ T cells, we conjugated the M290 antibody with saporin to produce an anti-mouse CD103-immunotoxin (M290-SAP). The M290 is a rat IgG2a monoclonal antibody that blocks interaction of CD103 with its ligand, E-cadherin (Karecla, P. I., et al. Eur J Immunol 25:852-856 (1995); Roberts, K., and P. J. Kilshaw, Eur J Immunol 23:1630-1635 (1993), which are incorporated by reference). To date, however, the M290 antibody has not been shown to deplete CD103-expressing cells in vivo.

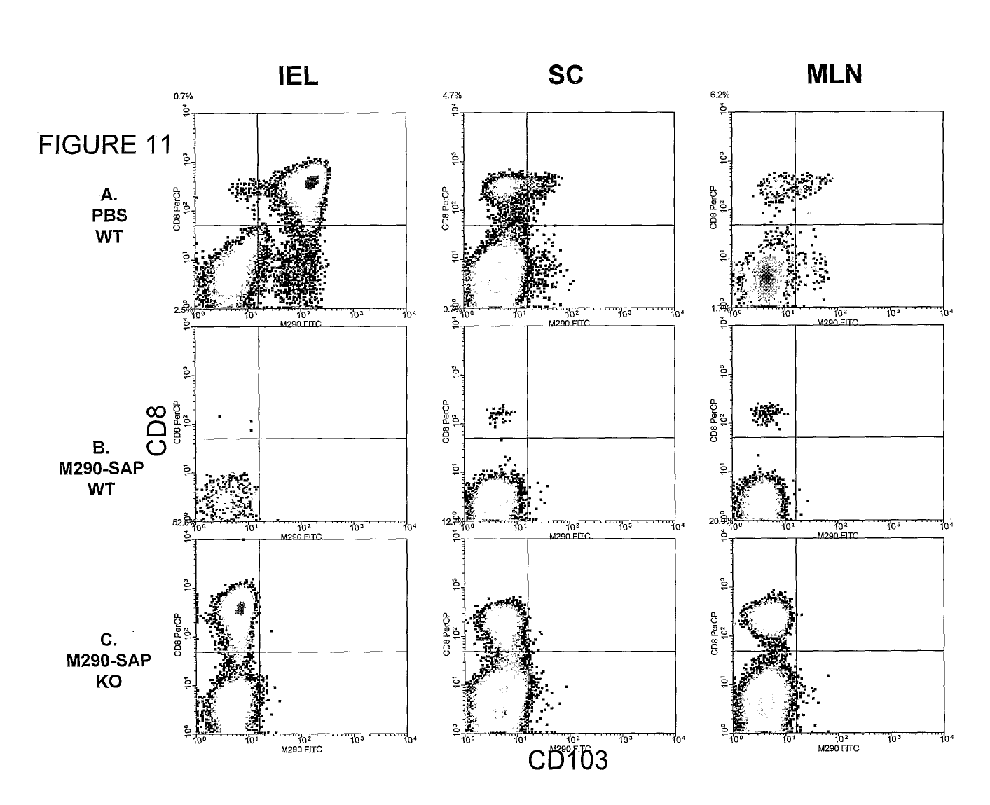

C57BL/6 mice were injected i.p. with either PBS (0.5 ml) as control only or with (A) or with 100 μg of the M290-MAP in 0.5 ml of PBS. After 3 days, cells were harvested from the intestinal epithelium (IEL), spleen or mesenteric lymph node (MLN), and subsequently subjected to two-color FACS® using mAbs to CD8 and CD103. Within three days, post injection, CD8+ T cells were depleted in all compartments examined including the intestinal epithelium, spleen, and MLN. Compared to PBS-dosed controls (FIG. 11A), CD8+ T cells in wild-type mice receiving the M290-MAP were dramatically reduced (FIG. 11B). In CD103 knockout mice, however, administration of M290-MAP into had no effect on total CD8+ cells (FIG. 11C).

What is claimed is:

1. A method of treating or preventing graft versus host disease (GVHD) in a host subject receiving a graft, said method comprising treating the graft to reduce the activity of CD103 in cells originating from the graft.

2. The method of claim 1, wherein the GVHD is acute GHVD.

3. The method of claim 2, wherein the host subject is a human.

4. The method of claim 2, wherein the graft is an isograft in relation to the host subject.

5. The method of claim 2, wherein the graft is an allograft in relation to the host subject.

6. The method of claim 2, wherein the graft is a xenograft in relation to the host subject.

7. The method of claim 2, wherein the GVHD in the host subject occurs in the intestine of the host subject.

8. The method of claim 7, wherein the GVHD in the host subject occurs in the intestinal epithelium.

9. The method of claim 2, wherein the cells originating from the graft comprise CD8+ T cells.

10. The method of claim 9, wherein treating the graft comprises reducing the ability of the CD8+ T cells to express CD103.

11. The method of claim 9, wherein reducing the ability of the CD8+ T cells to express CD103 comprises administering a composition comprising an antibody specific for CD103.

12. The method of claim 9, wherein reducing the ability of the CD8+ T cells to express CD103 comprises administering a composition that antagonizes transforming growth factor beta (TGF-β) upon the CD8+ T cells.

13. The method of claim 2, wherein the graft is treated prior to removal from a donor organism.

14. The method of claim 2, wherein the graft is treated after removal from the donor organism.

15. The method of claim 14, wherein the graft is treated after removal from a donor organism, but prior to transplantation.

16. The method of claim 14, wherein the graft is transplanted into the host subject before said treatment of the graft.

17. The method of claim 16, wherein the host subject is a human.

18. The method of claim 16, wherein the graft is an isograft in relation to the host subject.

19. The method of claim 16, wherein the graft is an allograft in relation to the host subject.

20. The method of claim 16, wherein the graft is a xenograft in relation to the host subject.

21. The method of claim 16, wherein the GVHD in the host subject occurs in the intestine of the host subject.

22. The method of claim 21, wherein the GVHD in the host subject occurs in the intestinal epithelium.

23. The method of claim 16, wherein the cells originating from the graft comprise CD8+ T cells.

24. The method of claim 23, wherein treating the graft comprises reducing the ability of the CD8+ T cells to express CD103.

25. The method of claim 23, wherein reducing the ability of the CD8+ T cells to express CD103 comprises administering a composition comprising an antibody specific for CD103.

26. The method of claim 23, wherein reducing the ability of the CD8+ T cells to express CD103 comprises administering a composition that antagonizes transforming growth factor beta (TGF-β) upon the CD8+ T cells.

27. The method of claim 16, wherein the graft is treated both before and after transplantation into the host subject.

28. A method of treating or preventing graft versus host disease (GVHD) in a host subject, said method comprising treating the host subject to reduce the ability of host cells to interact with CD103-expressing cells originating in the graft.

29. The method of claim 28, wherein the graft is transplanted into the host subject after treating the host subject.

30. The method of claim 28, wherein the graft is an isograft in relation to the host subject.

31. The method of claim 28, wherein the graft is an allograft in relation to the host subject.

32. The method of claim 28, wherein the graft is a xenograft in relation to the host subject.

33. The method of claim 28, wherein the GVHD in the host subject occurs in the intestine of the host subject.

34. The method of claim 33, wherein the GVHD in the host subject occurs in the intestinal epithelium.

35. The method of claim 28, wherein the CD103-expressing cells originating from the graft comprise CD8+ T cells.

36. The method of claim 28, wherein treating the host comprises reducing the ability of the host cells to express E-cadherin.

37. The method of claim 36, wherein treating the host comprises administering a composition comprising an antibody specific for E-cadherin.

38. The method of claim 36, wherein treating the host comprises administering an antagonist to transforming growth factor beta (TGF-β).

39. The method of claim 38, wherein the antagonist reduces expression levels of transforming growth factor beta (TGF-β).

40. The method of claim 28, further comprising treating the graft to reduce the activity of CD103 in cells originating from the graft.

41. The method of claim 28, wherein the graft is transplanted into the host subject before treating the host subject.

42. The method of claim 41, wherein the graft is an isograft in relation to the host subject.

43. The method of claim 41, wherein the graft is an allograft in relation to the host subject.

44. The method of claim 41, wherein the graft is a xenograft in relation to the host subject.

45. The method of claim 41, wherein the GVHD in the host subject occurs in the intestine of the host subject.

46. The method of claim 45, wherein the GVHD in the host subject occurs in the intestinal epithelium.

47. The method of claim 41, wherein the cells originating from the graft comprise CD8+ T cells.

48. The method of claim 41, wherein the CD103-expressing cells originating from the graft comprise CD8+ T cells.

49. The method of claim 41, wherein treating the host comprises reducing the ability of the host cells to express E-cadherin.

50. The method of claim 49, wherein treating the host comprises administering a composition comprising an antibody specific for E-cadherin.

51. The method of claim 49, wherein treating the host comprises administering an antagonist to transforming growth factor beta (TGF-β).

52. The method of claim 51, wherein the antagonist reduces expression levels of transforming growth factor beta (TGF-β).

53. The method of claim 41, further comprising treating the graft to reduce the activity of CD103 in cells originating from the graft.

54. A method of screening a graft to determine the likelihood that the graft will generate graft versus host disease (GVHD) after transplantation, said method comprising
a) determining the levels of CD103 in the graft prior to transplantation; and
b) comparing the determined levels of CD103 in the graft with normal levels of CD103 in similar tissue to determine a difference between the levels of CD103 in the graft versus normal levels of CD103,
wherein the difference indicates the likelihood that the graft will generate GVHD.

55. The method of claim 54, where the graft is treated to reduce the levels of CD103 prior to performing the screening method.

56. The method of claim 54, wherein the graft is treated to reduce the levels of CD103 after performing the screening method.

57. The method of claim 54, wherein determining the levels of CD103 in the graft are assessed in CD8+ T cells.

58. The method of claim 57, wherein determining the levels of CD103 comprise and immunoassay.

59. The method of claim 58, wherein the immunoassay comprises an antibody specific for CD103.

60. A method of screening compounds to treat graft versus host disease (GVHD), said methods comprising
a) administering a candidate compound to CD103; and
b) determining the level of binding between said candidate and CD103.

61. The method of claim 60, wherein the CD103 is in a cell-free environment.

62. The method of claim 60, wherein said CD103 is being expressed by a cell.
